(19) United States
(12) Patent Application Publication
(10) Pub. No.: US 2025/0339125 A1
(43) Pub. Date: Nov. 6, 2025

(54) ULTRASOUND PATCH

(71) Applicant: Becton, Dickinson and Company, Franklin Lakes, NJ (US)

(72) Inventors: Julio Agustin Isla Garcia, Irvine, CA (US); Andres S. Aguirre, Mission Viejo, CA (US); Rendle Lamarr Myles, JR., Covina, CA (US); Blake W. Axelrod, South Pasadena, CA (US); Timothy John Payne, Santa Ana, CA (US); Dylan Joseph Boyle, Huntington Beach, CA (US)

(21) Appl. No.: 19/265,875

(22) Filed: Jul. 10, 2025

Related U.S. Application Data
(63) Continuation of application No. PCT/US2024/011072, filed on Jan. 10, 2024.
(60) Provisional application No. 63/479,245, filed on Jan. 10, 2023.

(51) Int. Cl. A61B 8/00

(52) U.S. Cl. CPC A61B 8/4236; A61B 8/4281; A61B 8/4422

(57) ABSTRACT

A dual-function ultrasound device for ultrasonic scanning and use as a wearable ultrasonic patch includes an ultrasound transducer, an adherable couplant attached to the ultrasound transducer, and a frame containing an ultrasonic couplant. The frame is detachably coupled to the ultrasound transducer via the adherable couplant. A method of using a dual-function ultrasound device includes attaching an ultrasound transducer to a frame including an ultrasonic couplant, scanning, via the ultrasound transducer and the ultrasonic couplant, an area on a human body to identify a target location for ultrasound monitoring, detaching the frame including the ultrasonic couplant from the ultrasound transducer, and adhering the ultrasound transducer to the target location for ultrasound monitoring of the target location.

### CROSS-REFERENCE TO RELATED APPLICATION(S)

This application is a continuation of International Application No. PCT/US2024/011072, filed Jan. 10, 2024, entitled “ULTRASOUND PATCH,” which claims the benefit of U.S. Provisional Application No. 63/479,245, filed Jan. 10, 2023, and entitled “ULTRASOUND PATCH,” the disclosures of which are hereby incorporated by reference in their entireties.

### BACKGROUND

Currently, there is no test to indicate the status of the kidney in real time that can give physicians actionable information. While doppler ultrasound biomarkers, particularly the resistive index and the newly proposed VEXUS (Venous Excess Ultrasound Score), have been found to correlate with the occurrence of acute kidney injury (AKI), the measurement is time consuming and prone to error. A variety of wearable ultrasound devices have been proposed for long-term monitoring but suffer significant drawbacks. Many of the proposed devices do not include reusable ultrasound transducers, which adds a significant cost. Other devices do not protect reusable ultrasound transducers from biological contamination and, therefore, must be decontaminated prior to reuse. Additionally, proposed wearable ultrasound patches are not configured to scan a patient's body to identify a target area of interest prior to being affixed to a patient's body. As such, additional ultrasound devices may be required to identify a target area of interest prior to deployment of the ultrasound patch for long-term monitoring. This additional step can present challenges in busy, time-critical settings such as the operating room and intensive care unit.

### SUMMARY

In one aspect of the present disclosure, a dual-function ultrasound device for ultrasonic scanning and use as a wearable ultrasonic patch includes an ultrasound transducer, a detachable frame including an ultrasonic couplant coupled to the ultrasound transducer, and a protective barrier configured to isolate the ultrasound transducer from biological contamination.

In another aspect of the present disclosure, a dual-function ultrasound device for ultrasonic scanning and use as a wearable ultrasonic patch includes an ultrasound transducer, an adherable couplant attached to the ultrasound transducer, and a frame containing an ultrasonic couplant. The frame is detachably coupled to the ultrasound transducer via the adherable couplant.

In yet another aspect of the present disclosure, a device for use with an ultrasound transducer includes a frame, an ultrasonic couplant disposed in the frame, an adherable couplant disposed on the ultrasonic couplant. The ultrasonic couplant and the adherable couplant are capable of transmitting ultrasound energy. The adherable couplant is configured to interface with the ultrasound transducer. The ultrasonic couplant is configured to interface with the skin of a patient.

In yet another aspect of the present disclosure, a method of using a dual-function ultrasound device includes attaching an ultrasound transducer to a frame including an ultrasonic couplant, scanning, via the ultrasound transducer and the ultrasonic couplant, an area on a human body to identify a target location for ultrasound monitoring, detaching the frame including the ultrasonic couplant from the ultrasound transducer, and adhering the ultrasound transducer to the target location for ultrasound monitoring of the target location.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

FIG. 1 is a simplified cross-sectional exploded view of a dual-function ultrasound device.

FIG. 2 is a simplified side view of one example of an ultrasound transducer for use in the dual-function ultrasound device of FIG. 1.

FIG. 3 is a simplified bottom view of another example of an ultrasound transducer for use in the dual-function ultrasound device of FIG. 1.

FIG. 4 is a simplified cross-sectional view of one example of a disposable device for use with the dual-function ultrasound device of FIG. 1.

FIG. 5 is a simplified top view of one example of affixing mechanisms of a disposable device for use with the dual-function ultrasound device of FIG. 1

FIG. 6 is a simplified cross-sectional view of one example of an assembly of the affixing mechanisms of FIG. 5.

FIG. 7 is a simplified cross-sectional view of another example of an assembly of the affixing mechanisms of FIG. 5.

FIGS. 8A-8C are simplified cross-sectional views of a method for affixing the dual-function ultrasound device of FIG. 1 to skin.

FIG. 9 is a flow chart of a method for assembly and use of the dual-function ultrasound device of FIG. 1.

FIG. 10 is a perspective view of another dual-function ultrasound device.

FIG. 11A is an exploded view of the dual-function ultrasound device of FIG. 10.

FIG. 11B is an exploded view of the dual-function ultrasound device of FIG. 10

FIG. 11C is a perspective side view of the dual-function ultrasound device of FIG. 10 assembled for use.

FIG. 11D is a perspective bottom view of the dual-function ultrasound device of FIG. 10 assembled for use.

FIG. 12A is an exploded view of yet another dual-function ultrasound device.

FIG. 12B is a perspective view of the dual-function ultrasound device of FIG. 12A assembled for ultrasound scanning.

FIGS. 13A is perspective top view of an ultrasound transducer of FIG. 12A.

FIG. 13B is a perspective bottom view of an ultrasound transducer of FIG. 12A.

FIG. 14A is perspective top view of a frame of the dual-function ultrasound device of FIG. 12A.

FIG. 14B is perspective bottom view of a frame of the dual-function ultrasound device of FIG. 12A.

FIGS. 15A-15C are perspective views of a method of assembling a dual-function ultrasound device according to the present disclosure.

FIG. 16A is a perspective view of one example of packaging for the packaged device of FIG. 15A during removal of a packaging lid.

FIG. 16B is a perspective view of another example of packaging for the packaged device of FIG. 15A during removal of a packaging lid.

FIG. 16C is a perspective top view of the packaged device of FIG. 14B with a protective film layer.

FIG. 16D is a perspective top view of the packaged device of FIG. 14C during removal of the protective film layer.

FIGS. 17A and 17B are cross-sectional views of alternative methods of detaching a disposable device used for ultrasound scanning from an ultrasound device used for long-term monitoring and subsequent attachment of the ultrasound device to the skin of the patient for long- term monitoring.

FIG. 18 is a perspective view of an adhesive patch for securing dual-function ultrasound devices of the present disclosure to skin of a patient.

FIG. 19 is a flow chart of a method for assembly and use of the dual-function ultrasound devices of FIGS. 10 and 12.

### DETAILED DESCRIPTION

The present disclosure is directed to dual-function, wearable, ultrasound devices or patches that can be used to scan a patient's body to identify a target area of interest and to affix a reusable ultrasound transducer to a patient's body at the target area of interest for long-term monitoring. The disclosed ultrasound devices allow for the coupling of ultrasonic waves between the reusable ultrasound transducer and the patient's body during both the process of scanning the body to identify a target area of interest and during long-term monitoring at the target area of interest. The disclosed ultrasound devices can be used to scan a patient's body without a non-solid couplant (e.g., gel) or material that leaves a residue behind that could inhibit the effectiveness of permanent adhesives of the long-term wearable ultrasound patch. In some examples, the disclosed devices provide a sterile enclosure that acts as an engulfing and isolating barrier between the reusable ultrasound transducer and the patient's body to protect the reusable ultrasound transducer from biological contamination. The disclosed ultrasound devices are simple to deploy and require no additional devices or cleaning procedures, which is important in busy, time-critical settings such as the operating room and the intensive care unit.

The disclosed ultrasound devices are configured to collect information about a given parameter such as vessel pulsatility, blood flow within a vessel or vessel network, and tracking of other biological features such as organ motion or respiration rate. Such information can be utilized to infer and/or predict possible states of the human and/or animal body, like hypertension and hemorrhage, as well as other physiological variables, like stroke volume, stroke volume variations, and cardiac output. For example, the disclosed ultrasound devices can be used to track and measure the flow velocity profile in renal vessels, which can be used to compute known biomarkers such as the generally known resistive index and newly proposed VEXUS.

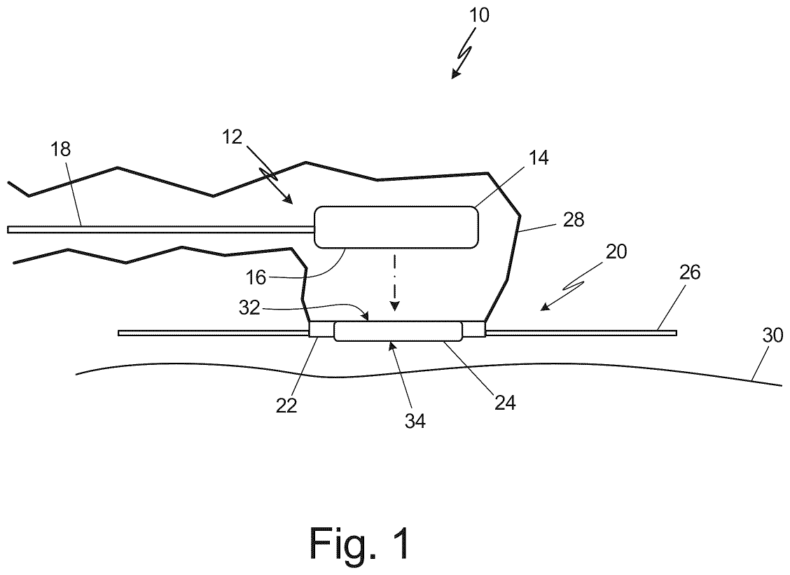

FIG. 1 is simplified cross-sectional exploded view of one example of a dual- function ultrasound device. FIG. 1 shows ultrasound device 10. Ultrasound device 10 includes reusable ultrasound transducer 12, including sensor head 14 (having face 16) and cable 18, and disposable device 20, including frame 22, ultrasonic couplant 24, adhesive strips 26, and protective barrier 28. Ultrasonic couplant 24 is contained in frame 22. Adhesive strips 26 extend outward from frame 22. Protective barrier 28 can extend outward from frame 22 and can engulf sensor head 14 and at least a portion of cable 18 of ultrasound transducer 12.

FIG. 1 shows the orientation of ultrasound device 10 relative to skin 30 of a patient's body prior to scanning or attachment for long-term monitoring. As used herein, long- term monitoring refers to monitoring for a period of time greater than a few minutes and which does not require operator assistance to maintain contact between ultrasound device 10 and skin 30. Ultrasound device 10 is a wearable device or patch that can be anchored to skin 30 for continuous or intermittent monitoring at the target location. Prior to use, reusable ultrasound transducer 12 is coupled to disposable device 20. Specifically, sensor head 14 can be attached to frame 22, as indicated by the arrow. Disposable device 20 is detachable from reusable ultrasound transducer 12 following use. Specifically, frame 22 can be detached from sensor head 14. Disposable device 20 can be disposed of following use. Reusable ultrasound transducer 12 can be used in subsequent applications with new disposable devices 20.

Ultrasound transducer 12 includes sensor head 14 and cable 18. Sensor head 14 has sensor face 16 disposed to interact with ultrasonic couplant 24. One or more cables 18 can extend from a side of sensor head 14 adjacent to sensor face 16. Ultrasound transducer 12 includes an array of transducer elements (not shown). Each transducer element of the array can comprise a piezoelectric material, such as lead zirconate titanate, capable of transmitting ultrasound pulses and detecting ultrasound pulses. The array of transducer elements can form a phased array. As a phased array, each transducer element in the array can pulse individually relative the other transducer elements in the array. A transducer controller controls the timing that each transducer element in the array emits an ultrasound pulse. The transducer controller can time and pattern when each transducer element emits a pulse such that the array can form one or more ultrasonic beams and sweep or steer the one or more ultrasonic beams without physically moving the position of ultrasound transducer 12 on a patient.

The transducer controller can be fully housed within a casing of ultrasound transducer 12. Cable(s) 18 can connect the transducer controller and ultrasound transducer 12 to a monitor unit. In other examples, at least part if not all of the transducer controller can be housed in the monitor unit and can be connected to ultrasound transducer 12 by cable(s) 18. Housing the transducer controller in the monitor unit can decrease the overall size and thickness of ultrasound transducer 12. Ultrasound transducer 12 can be relatively thin and flat in profile, with a thickness that is smaller than a width or diameter of ultrasound transducer 12. In some examples, ultrasound transducer has a height (thickness) to width ratio of less than half. Attaching ultrasound transducer 12 to a patient by an adhesive patch

is easier and more secure when ultrasound transducer 12 has a thin and flat profile. The array of transducer elements of ultrasound transducer 12 can be sized in length or diameter to cover one or more acoustic windows in a patient. An acoustic window of a patient is defined as an area on a patient where transmission of ultrasonic waves is not substantially attenuated in comparison to immediate surroundings. For example, an array of transducer elements of ultrasound transducer 12 can be sized in length to extend over at least two intercostal spaces of the patient.

In one embodiment, ultrasound transducer 12 is configured for measuring doppler flow signals of blood flow to organs in the abdomen of the patient, such as (but not limited to) the kidneys, the liver, the pancreas, and the spleen. In order to detect and measure the doppler flow signals of blood flow to an organ in the abdomen of the patient, such as the kidneys, the elements of ultrasound transducer 12 can have a low operating frequency between 0.5 MHz and 4.0 MHz and a size greater than one wavelength in soft tissue. With an operating frequency between 0.5 MHz and 4.0 MHz and a size greater than one wavelength in soft tissue, ultrasound transducer 12 can produce ultrasound beams that penetrate more than 15 cm into the patient, which is a sufficient depth to measure renal blood flow, hepatic blood flow, splenic blood flow, and pancreatic blood flow. The ultrasonic couplants, including adherable couplants and materials used therewith, disclosed herein are capable of transmitting ultrasound energy in the operating frequency range of ultrasound transducer 12 (i.e., frequency between 0.5 MHz and 4.0 MHz). In another embodiment, ultrasound transducer 12 is configured for measuring transcranial doppler signals from cerebral blood flow. The elements of the ultrasound transducer 12 may have similar parameters as in the above embodiment.

In other embodiments, the ultrasound transducer 12 is configured for visualization of internal body structures including vessels, organs, and muscles. In this configuration the structure of interest may be superficial or deep under the skin. The size and depth of the structure of interest will determine the appropriate ultrasound transducer frequency of operation to achieve the needed resolution.

In some embodiments, ultrasound transducer 12 may be configured to achieve a combination of both structural visualization and vessel blood flow measurements.

Disposable device 20 can be used with ultrasound transducer 12 to scan a patient's body to identify a target area of interest for long-term monitoring and can provide a long-term adherable couplant layer for use with ultrasound transducer 12 for long-term monitoring. As described further herein, disposable device 20 can be pre-assembled, packaged, and stored for use with ultrasound transducer 12 when needed. Frame 22 includes ultrasonic couplant 24. Ultrasonic couplant 24 provides a coupling medium or interface between sensor face 16 of sensor head 14 and skin 30 and is capable of transmitting ultrasound energy therebetween. Frame 22 can extend around a perimeter of ultrasonic couplant 24 such that a top surface 32 of ultrasonic couplant 24 facing sensor face 16 of sensor head 14 is exposed and a bottom surface 34 of ultrasonic couplant 24 facing skin 30 is exposed. Ultrasonic couplant 24 can be a solid-like couplant material capable of sliding over skin 30. Preferentially, ultrasonic couplant 24 is formed of a material that can slide over skin 30 without leaving a residue. Ultrasonic couplant 24 can be formed of a material having a slippery surface configured to slide over the skin with minimal resistance. Ultrasonic couplant 24 can include, for example, agar-based couplants, water-based couplants, alcohol-based couplants, and/or hydrogels. Hydrogels can be molded into and will maintain solid shapes and have very low friction over surfaces such as skin. Typical hydrogel coefficients of friction are 0.1 to 0.001. Ultrasonic couplant 24 can have a coefficient of friction of less than 0.1 and, preferably, less than 0.03. Ultrasonic couplant 24 can protrude from a lower surface of frame 22 to interface with skin 30. Bottom surface 34 of ultrasonic couplant 24 can have a planar surface with rounded edges, or a convex spherical or curved surface to promote the sliding motion of ultrasound device 10 over skin 30 during scanning. Unlike liquids, ultrasonic couplant 24 does not deform substantially under external forces, however, ultrasonic couplant 24 can be a flexible material capable of substantially conforming to a surface of skin 30 to displace air or minimize air pockets between skin 30 and ultrasonic couplant 24. In some examples, a wetting agent (e.g., water) can be used with ultrasonic couplant 24 to promote the sliding motion and/or to displace air between skin 30 and ultrasonic couplant 24. Preferably, wetting agents include materials that do not leave a residue on skin 30 or do not require cleaning of skin 30 for removal. Top surface 32 of ultrasonic couplant 24 can be planar to provide effective contact between ultrasonic couplant 24 and sensor face 16. Ultrasonic couplant 24 can be stored with a seal or film cover (not shown) on both interfacing surfaces 32 and 34. A seal on top surface 32 of ultrasonic couplant 24 can be removed prior to assembly with sensor head 14. A seal on bottom surface 36 of ultrasonic couplant 24 can be removed prior to performing an ultrasound scan on a patient's body.

Frame 22 can be shaped or configured, as further described herein, to retain ultrasonic couplant 24 in frame 22. Ultrasonic couplant 24 can be shaped, as further described herein, to be retained in frame 22. Frame 22 and ultrasonic couplant 24 can have any shape or configuration and are not limited to the shapes and configurations illustrated. As disclosed further herein, each of frame 22 and ultrasonic couplant 24 can have a substantially rectangular cross-sectional shape, however, other shapes, including circular and oblong shapes, are contemplated. In some examples, walls of frame 22 and ultrasonic couplant 24 can be tapered inward toward bottom surface 34 to retain ultrasonic couplant 24.

A total thickness of ultrasonic couplant 24 can be minimized to a thickness that provides for effective movement of ultrasound device 10 across the skin of a patient and retention of ultrasonic couplant 24 in frame 22. As further described herein, in some examples, ultrasonic couplant 24 can be formed of two or more layers. The two or more layers can be formed of the same or different couplant materials capable of transmitting ultrasound energy.

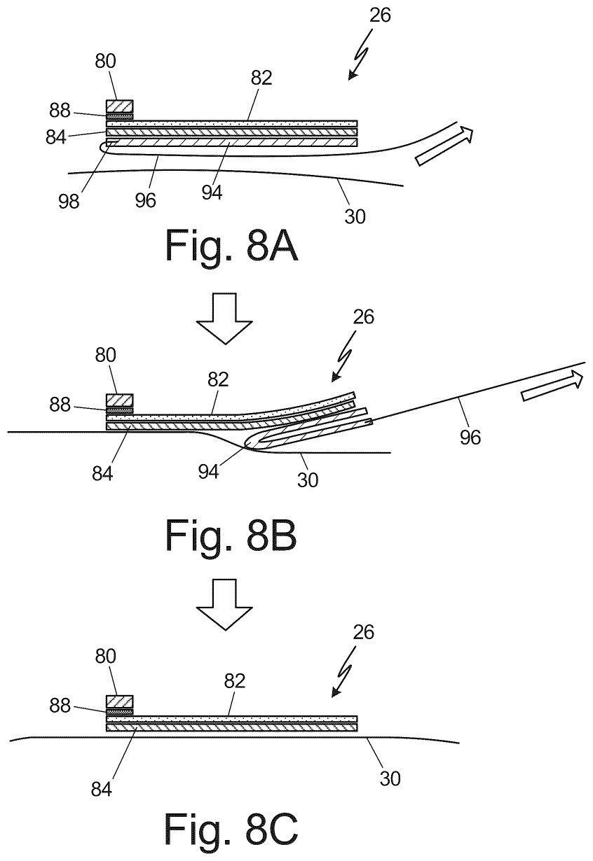

Adhesive strips 26 can be affixed to frame 22. Adhesive strips 26 extend outward from frame 22 generally parallel to a bottom surface of frame 22. As described further herein, separate adhesive strips 26 can be provided on each of four sides of a rectangular frame 22. A cover layer (shown in FIG. 8A) can cover an adhesive surface of adhesive strips 26 facing skin 30 during a scanning procedure such that ultrasound device 10 is free to move across skin 30. Once the target area of interest has been identified, the cover layer can be removed to anchor ultrasound device 10 to skin 30 for long-term monitoring.

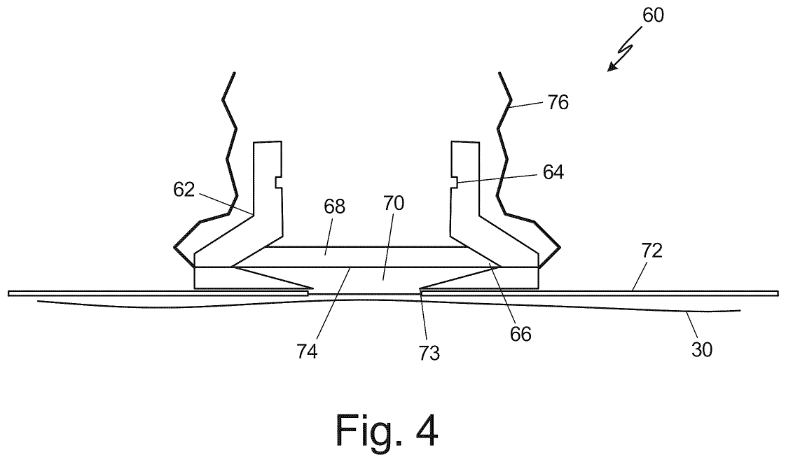

Protective barrier 28 can protect or isolate ultrasound transducer 12 from biological contamination during both the scanning procedure and during long-term monitoring. Protective barrier 28 can be affixed to frame 22. Protective barrier 28 can include a protective sheath that extends outward from frame 22 around sensor head 14 and at least a portion of cable 18 of ultrasound transducer 12. Protective barrier 28 can include, for example, a plastic sleeve or sheath. Protective barrier 28 can additionally include a disposable layer (shown, for example, in FIG. 4) extending across sensor face 16 of sensor head 14 such that protective barrier 28 fully engulfs sensor head 14 and a portion of cable 18 to further protect ultrasound transducer 12 from biological contamination. As such, ultrasound transducer 12 can be reused in subsequent applications with minimal or no cleaning.

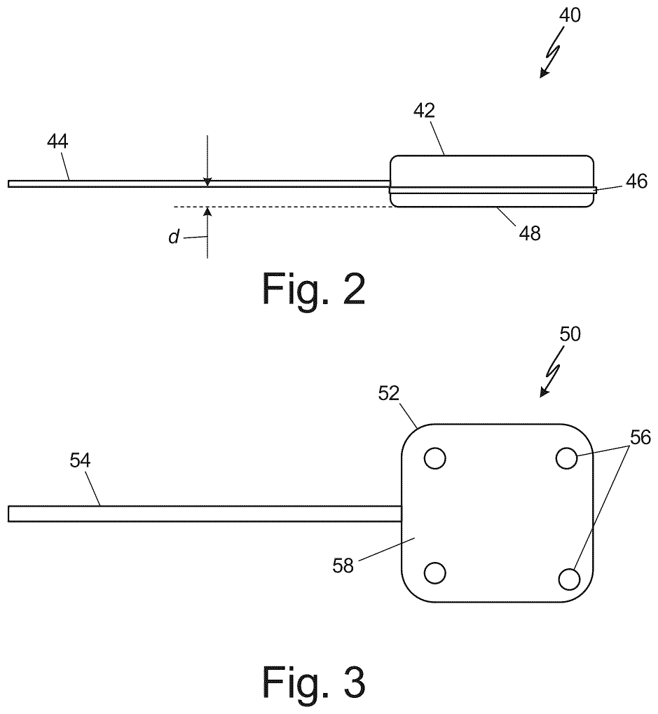

FIG. 2 is a simplified side view of one example of an ultrasound transducer for use in ultrasound device 10 of FIG. 1. FIG. 2 shows ultrasound transducer 40, sensor head 42, cable 44, attachment mechanism 46, and sensor face 48. FIG. 3 is a simplified bottom view of another example of an ultrasound transducer for use in ultrasound device 10 of FIG. 1. FIG. 3 shows ultrasound transducer 50, sensor head 52, cable 54, attachment mechanism 56, and sensor face 58. FIG. 4 is a simplified cross-sectional view of one example of a disposable device for use with ultrasound device 10 of FIG. 1. FIG. 4 shows disposable device 60, frame 62, attachment mechanism 64, recess 66, ultrasonic couplant layers 68 and 70, adhesive strips 72, protective barriers 74 and 76, and skin 30. FIGS. 2-4 are discussed together.

As described with respect to FIG. 1, prior to operation of ultrasound device 10, reusable ultrasound transducer 12 is coupled to disposable device 20. Specifically, sensor head 14 is attached to frame 22. FIGS. 2 and 3 illustrate alternative mechanisms for attaching sensor head 14 to frame 22. Each of disclosed attachment mechanisms 46 and 56 provide a means for retaining sensor head 14 in frame 22 and, specifically, for maintaining an effective interface between sensor face 16 of sensor head 14 and ultrasonic couplant 24. Attachment mechanisms 46 and 56 illustrated in FIGS. 2 and 3 are examples of attachment mechanisms that could be used to secure sensor head 14 to frame 22 and provide an effective interface between sensor head 14 and ultrasonic couplant 24. Attachment mechanisms are not limited to the examples illustrated in FIGS. 2 and 3. Other attachment mechanisms are contemplated. In each of the disclosed examples, cables 18, 44, 54 can extend from a side of the respective sensor head 14, 42, 52 adjacent to the respective sensor face 16, 48, 58 and can be offset from the respective sensor face 16, 48, 58 by a distance d (shown in FIG. 2) to provide additional space for the respective sensor head 14, 42, 52 to attach to frame 22 (or 62). In some examples, sensor head 14, 42, 52 can sit in frame 22 (or 62) such that frame 22 (or 62) extends around a perimeter of sensor head 14, 42, 52.

FIG. 2 shows ultrasound transducer 40 having sensor head 42. Sensor head 42 includes attachment mechanism 46. Attachment mechanism 46 is a ridge that protrudes from a side of sensor head 42 adjacent face 48. Face 48 is configured to couple to ultrasonic couplant 24 in frame 22. Attachment mechanism 46 can extend around a full or partial perimeter of sensor head 42. Attachment mechanism 46 is configured to be received in a corresponding attachment mechanism in frame 22 (shown in FIG. 4). For example, attachment mechanism 46 can be received in a groove of corresponding size and shape in frame 22. In some examples, attachment mechanism 46 can be received in a corresponding groove in frame 22 via a sliding fit or a snap fit, depending on the configuration of frame 22. In some examples, frame 22 can include one or more attachment features (e.g., clasp, hook, catch, or the like) configured to grasp attachment mechanism 46 and retain sensor head 42. Attachment mechanism 46 provides a temporary means for attaching sensor head 42 to frame 22. Once use of ultrasound device 10 is complete, sensor head 42 can be detached from frame 22.

FIG. 3 shows ultrasound transducer 50 having sensor head 52. FIG. 3 shows a bottom view of sensor head 52. Sensor head 52 can be substantially similar to sensor head 42 with the replacement of attachment mechanism 46 with attachment mechanism 56. Attachment mechanism 56 can be positioned on sensor face 58 of sensor head 52. Sensor face 58 is configured to interface with frame 22 and ultrasonic couplant 24. Attachment mechanisms 56 are disposed adjacent to one or more outer edges of frame 22 or a perimeter of sensor face 58 such that attachment mechanisms 56 do not interfere with a connection between sensor face 58 and ultrasonic couplant 24. In some examples, sensor face 58 can include an edge or perimeter that is recessed from a central portion of sensor face 58 configured to contact ultrasonic couplant 24. Attachment mechanisms 56 can be disposed in the recessed portion of sensor face 58. Attachment mechanisms 56 can be, for example, studs, holes, and/or magnets configured to attach sensor head 52 to frame 22. For example, attachment mechanisms 56 can include studs that protrude from sensor face 58 of sensor head 52 and that can be received in corresponding holes in frame 22.

Alternatively, attachment mechanisms 56 can include holes, which can receive corresponding studs of frame 22. Sensor head 52 and frame 22 can be attached, for example, by a press fit. Alternatively, attachment mechanisms 56 can include magnets or magnetic elements, which can engage corresponding magnetic elements or magnets of frame 22 to secure sensor head 52 to frame 22. Once use of ultrasound device 10 is complete, sensor head 52 can be detached from frame 22.

FIG. 4 shows one example of a disposable device that can be configured for use with ultrasound device 10. FIG. 4 shows disposable device 60 with frame 62 for use with ultrasound transducer 40 (shown in FIG. 2). Frame 62 is configured for attachment to sensor head 42 with attachment mechanism 46. FIG. 4 is not drawn to scale. Frame 62 includes attachment mechanism 64. Attachment mechanism 64 is a groove configured to receive corresponding attachment mechanism 46 (ridge) of sensor head 42 (shown in FIG. 2). In some examples, attachment mechanism 46 can be received in attachment mechanism 64 via a sliding fit or a snap fit, depending on the configuration of frame 62. For example, frame 62 can be configured to receive sensor head 42 by sliding a front end of sensor head 42 (opposite cable 44) into frame 62 and attachment mechanism 46 (ridge) into corresponding attachment mechanism 64 (groove), or walls of frame 62 can be flexible to allow sensor head 42 to be pressed into frame 62 such that attachment mechanism 46 (ridge) is received in attachment mechanism 64 (groove) with a snap fit. Frame 62 is configured to receive sensor head 42 such that sensor head 42 sits in frame 62 and frame 62 extends around a perimeter of sensor head 42. In other examples, frame 62 can include attachment mechanisms corresponding to attachment mechanisms 56 on sensor head 50.

Frame 62 can include one or more layers of ultrasonic couplant. As illustrated, frame 62 includes ultrasonic couplant layer 68 configured to interface with sensor head 42 and ultrasonic couplant layer 70 configured to interface with skin 30. Ultrasonic couplant layer 68 is disposed above ultrasonic couplant layer 70 in frame 62 and can protect sensor head 42 from biological contamination. Ultrasonic couplant layer 70 is formed of a material consistent with ultrasonic couplant 24 as previously described. Ultrasonic couplant layer 68 can be formed of the same material as ultrasonic couplant layer 70 or a different material capable of transmitting ultrasonic energy. In some examples, ultrasonic couplant layer 70 can have a thickness less than ultrasonic couplant layer 68. Frame 62 can be shaped to retain ultrasonic couplant layers 68 and 70. For example, as illustrated in FIG. 4, frame 62 can include recess 66 formed by angled walls of frame 62 configured to retain ultrasonic couplant layers 68 and 70 in frame 62. Frame walls can taper outward from a top surface of ultrasonic couplant layer 68 to recess 66 to retain ultrasonic couplant layer 68 upon removal of sensor head 42. Frame walls can taper inward from recess 66 to bottom opening 73 in frame 62 to retain ultrasonic couplant layer 70 (i.e., prevent ultrasonic couplant layer 70 from falling out bottom opening 73 in frame 62). Ultrasonic couplant layers 68 and 70 can have shapes corresponding to the frame walls. Ultrasonic couplant layer 68 can taper outward from a top surface configured to face sensor head 42 to a bottom surface facing ultrasonic couplant layer 70. Ultrasonic couplant layer 70 can taper inward from a top surface facing ultrasonic couplant layer 68 to a bottom surface configured to face skin 30. Bottom opening 73 in frame 62 and/or ultrasonic couplant layer 70 can be configured to allow a portion of ultrasonic couplant layer 70 to protrude through frame 62 to interface with skin 30. Other frame configurations can be used to retain the ultrasonic couplant. Mechanisms for ultrasonic couplant retention are not limited to the frame configuration or shape shown in FIG. 4.

In some examples, ultrasonic couplant layers 68 and 70 can be separated by protective barrier 74. Protective barrier 74 can be a protective film capable of transmitting ultrasound energy. Protective barrier 74 can extend fully between ultrasonic couplant layers 68 and 70 such that ultrasonic couplant layer 68 is fully separated from ultrasonic couplant layer 70. In some examples, protective barrier 74 can extend into frame 62. Protective barrier 74 can be provided to further protect sensor face 48 of sensor head 42 from biological contamination contacting ultrasonic couplant layer 70.

Protective barrier 76 is a protective sheath that can be affixed to frame 62 and can extend around sensor head 42 and at least a portion of cable 44 of ultrasound transducer 40. Together, protective barrier 74 and protective barrier 76 can engulf sensor head 42 and at least a portion of cable 44 of ultrasound transducer 40 to protect ultrasound transducer 40 from biological contamination. In alternative examples, frame 62 can include a single ultrasonic couplant layer and protective barrier 74 can be eliminated. In some examples, protective barrier (sheath) 76 can be attached to sensor head 42. For example, protective barrier 76 can be attached to a perimeter of a sensor face 48 such that it does not interfere with ultrasonic couplant layer 68 and can extend around sensor head 42 and at least a portion of cable 44. In such examples, protective barrier 76 can be received in frame 62.

Adhesive strips 72 are attached to frame 62. As illustrated in FIG. 4, adhesive strips 72 can be attached to the bottom surface of frame 62. For example, adhesive strips 72 can be adhered to the bottom surface of frame 62 by an adhesive. As previously disclosed, a cover layer (e.g., shown in FIG. 8A) can be disposed on an adhesive side of adhesive strips 72 (facing skin 30) to allow ultrasound device 10 to freely move across skin 30 during an ultrasound scanning procedure. Once a target location has been identified for long-term monitoring, the cover layer can be removed and ultrasound device 10 can be affixed to skin 30.

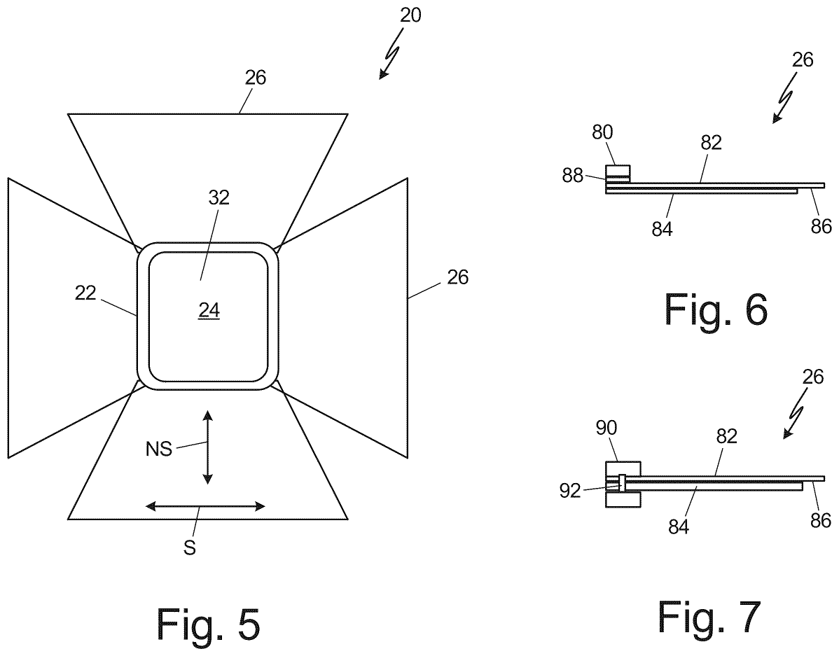

FIG. 5 is a simplified top view of disposable device 20 for use with ultrasound device 10 of FIG. 1. FIG. 5 illustrates the mechanisms for affixing ultrasound device 10 to a patient for long-term monitoring. Disposable device 20, frame 22, ultrasonic couplant 24, and adhesive strips 26 are shown. Frame 22 houses ultrasonic couplant 24 and extends around a perimeter of ultrasonic couplant 24. Top surface 32 of ultrasonic couplant 24 is shown. Top surface 32 of ultrasonic couplant 24 is configured to interface with sensor head 14 of ultrasound transducer 12. Frame 22 can have four sides. Adhesive strips 26 can extend outward from frame 22 and generally parallel to a bottom surface of frame 22. One adhesive strip 26 can extend outward from each side of frame 22 such that four adhesive strips 26 surround frame 22. As previously disclosed, the shape of frame 22 is not limited to the shape disclosed. In other examples, frame 22 can have a circular or oblong cross-sectional shape. The number and shape of adhesive strips 26 can be optimized to secure ultrasound device 10 to a patient for long-term monitoring. Adhesive strips 26 can be cut in a way that allows conformability to a curved surface (i.e., body of a patient).

Adhesive strips 26 can be formed, for example, of a fabric material having an adhesive surface. Adhesive strips 26 can be formed of a one-way stretch material configured to promote adhesion to skin 30 while maintaining a pulling force necessary to secure ultrasound device 10 at the target location. For example, adhesive strips 26 can be configured such that no stretch occurs in a radial dimension with respect to a center of frame 22 (and sensor head 14), indicated as a direction of no stretch NS. The absence of stretching in this direction helps maintain a pulling force necessary to maintain the position of ultrasound device 10 and can reduce the likelihood that ultrasound device 10 is inadvertently pulled away from the target area of interest during application of adhesive strips 26 or during long-term monitoring. Stretching can occur in a lateral dimension (opposite of the direction of no stretch NS), indicated as the direction of stretch S. Stretching in this direction can increase a surface area of skin 30 covered by adhesive strips 26 and can improve adhesion.

FIG. 6 is a simplified cross-sectional view of one example of an assembly of the affixing mechanisms of FIG. 5. Frame 80, adhesive strip 26, frame coupling layer 82, adhesive layer 84, non-adhesive portion 86, and frame adhesive 88 are shown. Frame 80 can be substantially similar to frame 22 of FIGS. 1 and 5 or frame 62 of FIG. 4, or a variation thereof as disclosed herein. FIG. 6 shows a single edge of frame 80 and single adhesive strip 26 extending outward from frame 80. Adhesive strip 26 can include frame coupling layer 82, which is a non-adhesive layer. Frame coupling layer 82 can be, for example, a fabric layer having one-way stretch as disclosed with respect FIG. 5. Adhesive layer 84 is disposed on an underside of frame coupling layer 82. Adhesive layer 84 can be formed of an adhesive material suitable for securing frame coupling layer 82 to skin. Adhesive layer 84 can be, for example a removable pressure-sensitive adhesive suitable for medical use as known in the art. Adhesive layer 84 can extend from an edge of frame coupling layer 82 aligned with the edge of frame 80 to secure a bottom surface of frame 80 to the patient's skin. Adhesive layer 84 can extend a partial length of frame coupling layer 82 such that an edge of frame coupling layer 82 opposite frame 80 is not covered by adhesive layer 84, thereby forming non-adhesive portion 86. Non-adhesive portion 86 can form a tab which is unsecured to a patient's skin, and which can be used facilitate removal of adhesive strips 26 from the skin upon completion of use of ultrasound device 10.

Adhesive strip 26 can be secured to a bottom side of frame 80. Adhesive strip 26 can be secured to the bottom side of frame 80 by frame adhesive 88. Frame adhesive 88 is disposed between the bottom surface of frame 80 and a top surface of frame coupling layer 82. Frame adhesive 88 can be any adhesive material capable of securing adhesive strip 26 to frame 80 for long-term use. Frame adhesive 88 can be a permanent adhesive. Both frame 80 and adhesive strip 26 are disposable and generally do not need to be detached.

FIG. 7 is a simplified cross-sectional view of another example of an assembly of the affixing mechanisms of FIG. 5. Frame 90 with attachment mechanism 92 and adhesive strip 26 with frame coupling layer 82, adhesive layer 84, non-adhesive portion 86, and frame adhesive 88 are shown. Frame 90 can be substantially similar to frame 22 shown in FIGS. 1 and 5 and described with respect thereto, or a variation thereof as disclosed herein. FIG. 7 shows a single edge of frame 90 and single adhesive strip 26 extending outward from frame 90. Adhesive strip 26 is mechanically fixed between portions of frame 90 by attachment mechanisms 92. A plurality of attachment mechanisms 92 can be disposed along the side of frame 90 and can capture and retain adhesive layer 26. For example, frame 90 can be formed of two portions joined by studs protruding from one portion and received in holes of the adjacent portion. The studs can extend through holes in adhesive strip 26 to retain adhesive strip 26 between frame portions. Studs can be retained with a press fit. In other examples, attachment mechanisms 92 can be threaded fasteners, rivets, sutures, or similar fastening mechanism known in the art.

FIGS. 8A-8C are simplified cross-sectional views of a method for affixing ultrasound device 10 of FIG. 1 to skin using adhesive strips 26. Adhesive strip 26, frame coupling layer 82, adhesive layer 84, cover layer 94, release strip 96, anchor 98, frame 80, frame adhesive 88, and skin 30 are shown. Adhesive strip 26 is assembled with frame 80 via frame adhesive 88 as shown in FIG. 6 and described with respect thereto. Adhesive strip 26 includes frame coupling layer 82 and adhesive layer 84 as described with respect to FIG. 6. Adhesive strip 26 additionally includes cover layer 94 and release strip 96. Cover layer 94 is disposed on adhesive layer 84. Release strip 96 is attached to cover layer 94 at anchor 98. FIGS. 8A-8C are discussed together.

Cover layer 94 can be a release liner or backing paper configured to cover adhesive layer 84 prior to affixing adhesive strip 26 to skin 30 and configured to peel off of adhesive layer 84 cleanly or without removing adhesive layer 84. Cover layer 94 can protect adhesive layer 84 and can slide against skin 30 without interrupting movement of disposable device 20 during the ultrasound scanning procedure.

Release strip 96 is disposed on an underside of cover layer 94 adjacent to skin 30 and is configured to facilitate removal of cover layer 94. Release strip 96 can be attached to an inner edge of cover layer 94 adjacent to frame 80 via anchor 98. For example, anchor 98 can be fixed to an inner surface or outer surface of cover layer 94. Release strip 96 can have a length greater than a length of adhesive strip 26 such that release strip 96 extends outward from adhesive strip 26 to provide ease of access to release strip 96. Release strip 96 can be a strip of material or string or the like capable of pulling cover layer 94 away from adhesive layer 84. Release strip 96 can be configured to pull cover layer 94 away from adhesive layer 84 without tearing cover layer 94. Release strip 96 can have a width less than cover layer 94. Release strip 96 can be formed of a material capable of sliding against skin 30 during the ultrasound scanning procedure without interrupting movement of disposable device 20. The arrow in FIG. 8A indicates the direction in which release strip 96 is pulled to remove cover layer 94.

FIG. 8B illustrates the step of removing cover layer 94 from adhesive layer 84. As shown in FIG. 8B, release strip 96 is pulled in the direction of the arrow, which removes cover layer 94 starting at anchor 98. As cover layer 94 is removed, adhesive layer 84 is allowed to contact skin 30. A user can apply pressure to ultrasound device 10 to hold frame 80 in place during removal of cover layer 94.

FIG. 8C illustrates adhesive strip 26 secured to skin 30 following the removal of cover layer 94. As previously discussed, multiple adhesive strips 26 can be secured to a frame of disposable device 20. Each adhesive strip 26 can be adhered to skin 30 independently in separate steps. Alternatively, a single pulling strip can be adhered to all adhesive strips 26 and configured to remove cover layers 94 from each adhesive strip 26 simultaneously. For example, an end of release strip 96 opposite anchor 98 can be connected to ends of other release strips 96 associated with other adhesive strips 26. The ends can be connected at a single joining feature such that all ends of release strips 96 can be pulled simultaneously by pulling the single joining feature.

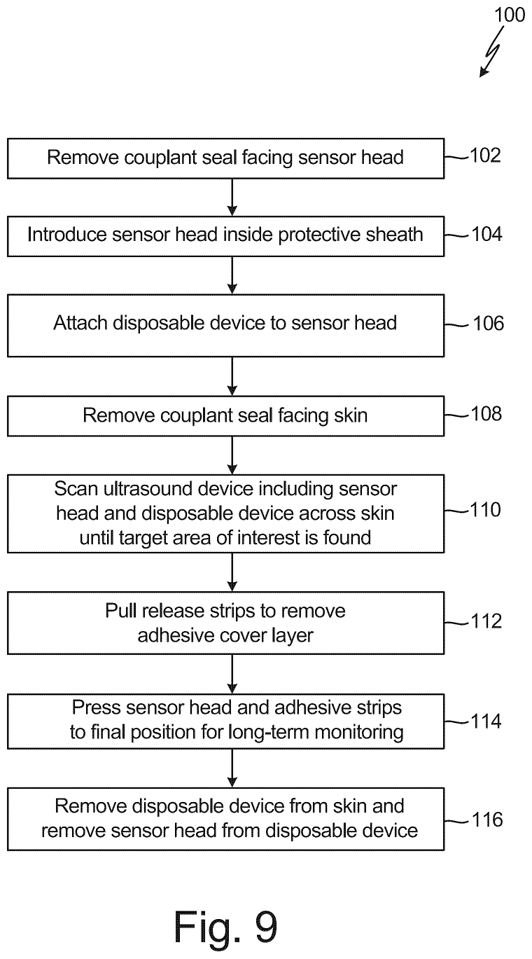

FIG. 9 is a flow chart of a method for assembly and use of ultrasound device 10 of FIG. 1. FIG. 9 shows method 100. Step 102 includes removing a seal from a surface of ultrasonic couplant 24 facing sensor head 14. Disposable device 20 can be packaged with one or more seals or film covers protecting exposed surfaces of ultrasonic couplant 24. The seal can extend across top surface 32 of ultrasonic couplant 24. The seal can include a tab and/or patterned cut for case of removal as described further herein.

Step 104 includes introducing sensor head 14 of ultrasound transducer 12 inside protective barrier 28 (i.e., protective sheath). Protective barrier 28 can be pulled up around sensor head 14 and cable 18 of ultrasound transducer 12. Protective barrier 28 can extend a length of cable 18 sufficient to protect ultrasound transducer from biological contamination. Protective barrier 28 can be connected to frame 22 as illustrated or can be connected to sensor head 14 as disclosed.

Step 106 includes attaching sensor head 14 of ultrasound transducer 12 to disposable device 20. Specifically, step 106 includes attaching sensor head 14 to frame 22. Sensor head 14 can be mechanically or magnetically attached to frame 22 with attachment mechanisms including but not limited to the attachment mechanisms described with respect to FIGS. 2-4.

Step 108 includes removing a seal from a surface of ultrasonic couplant 24 facing skin 30. As previously discussed, disposable device 20 can be packaged with one or more seals or film covers protecting exposed surfaces of ultrasonic couplant 24. The seal can extend across bottom surface 34 of ultrasonic couplant 24. The seal can include a tab and/or patterned cut for case of removal as described further herein.

Step 110 includes scanning the assembled ultrasound device 10, which includes sensor head 14 and disposable device 20, across skin 30 until a target area of interest is identified. Step 110 includes transmitting ultrasound energy from ultrasound transducer 12 and receiving transmitted ultrasound energy from the area scanned as known in the art. In some examples, step 110 can optionally include adding a couplant or wetting agent to skin 30 and/or to ultrasonic couplant 24 to facilitate the transmission of the ultrasound energy and sliding of ultrasonic couplant 24 on skin 30. Preferably, the couplant or wetting agent is a material that can dry without leaving a residue and does not interfere with adhering ultrasound device 10 to skin 30 once the target area of interest is located.

Step 112 includes pulling one or more release strips 96 to remove cover layers 94 from adhesive layers 84 of adhesive strips 26, as disclosed with respect to FIGS. 8A-8C. Once the target area of interest is located, ultrasound device 10 can be held in position and one or more release strips 96 can be pulled to expose adhesive layers 84 of adhesive strips 26.

Step 114 includes pressing sensor head 14 and adhesive strips 26 onto skin 30 for long-term monitoring. Adhesive strips 26 secure ultrasonic couplant 24 against skin 30 for long-term ultrasound monitoring. Ultrasound device 10 can remain secured to skin 30 as a wearable patch until long-term ultrasound monitoring is completed.

Step 116 includes removing disposable device 20 from skin 30 and removing ultrasound transducer 12 from disposable device 20 once long-term ultrasound monitoring is complete. Disposable device 20 can be removed from skin 30 by removing adhesive strips 26 from skin 30. In some examples, a non-adhesive tab on ends of adhesive strips 26 can be used to facilitate removal of adhesive strips 26 as described with respect to FIGS. 6 and 7. Ultrasound transducer 12 can be removed from disposable device 20 by releasing attachment mechanisms coupling sensor head 14 to frame 22. Disposable device 20 can be disposed of. Ultrasound transducer 12 can be reused in subsequent applications with new disposable devices 20. Protective barrier 28 can limit an amount of cleaning required or eliminate the need for cleaning ultrasound transducer 12 for subsequent use. Protective barrier 28 can be disposed of.

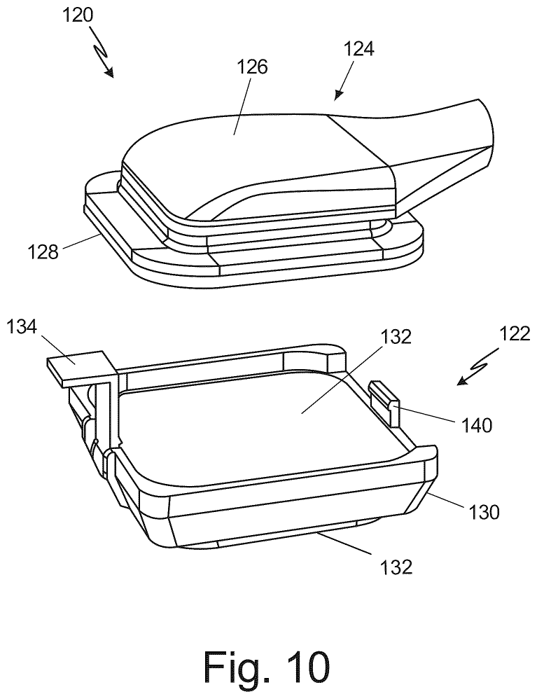

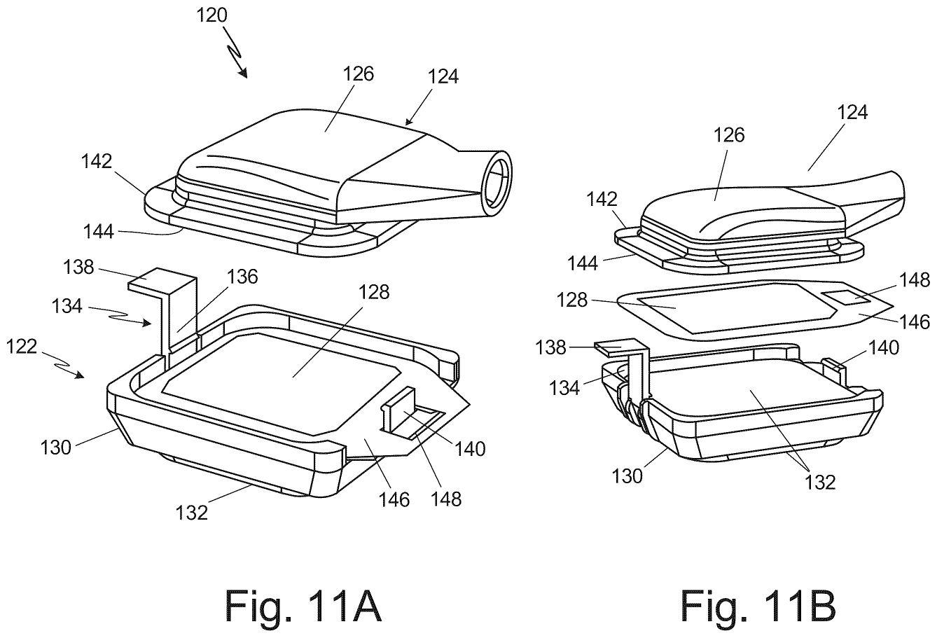

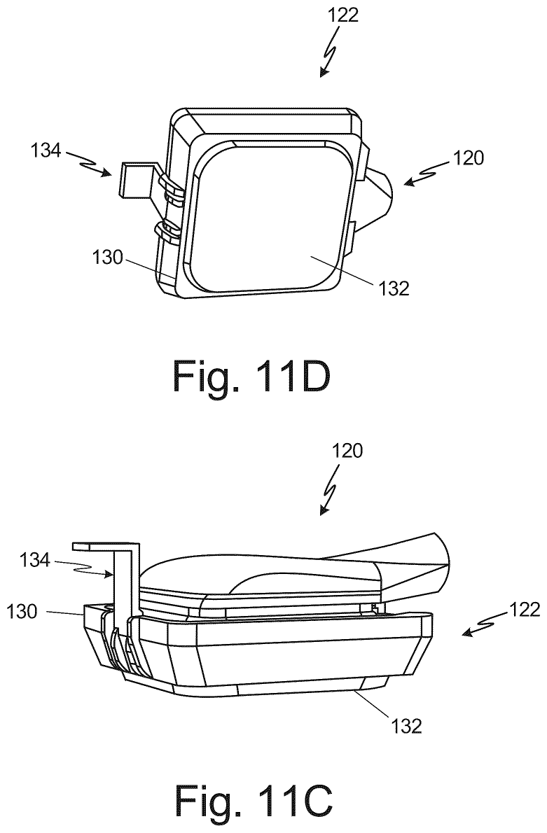

FIG. 10 is a perspective view of another dual-function ultrasound device having an ultrasound transducer coupled to a disposable device for ultrasound scanning and coupled to an adherable couplant for long-term ultrasound monitoring. FIG. 10 shows ultrasound device 120 and disposable device 122. FIG. 11A is a perspective view of a portion of ultrasound device 120 of FIG. 10 prior to assembly and disposable device 122. FIG. 11B is a partially exploded view of ultrasound device 120 of FIG. 11A and disposable device 122. FIG. 11C is a perspective side view of ultrasound device 120 and disposable device 122 of FIG. 10 assembled for use. FIG. 11D is a perspective bottom view of ultrasound device 120 and disposable device 122 of FIG. 10 assembled for use. Ultrasound device 120, disposable device 122, ultrasound transducer 124, sensor head 126, adherable couplant 128, frame 130, ultrasonic couplant 132, ejection element 134, front clip 136, ejection tab 138, back clip 140, flange 142, sensor face 144, film liner 146, and opening 148 are shown. FIGS. 11A-11D are discussed together.

Ultrasound device 120 is a dual function ultrasound device configured for use with disposable device 122 for ultrasound scanning to identify a target area of interest and configured for use alone (without disposable device 122) as an ultrasound patch for long-term ultrasound monitoring. Ultrasound device 120 assembled with disposable device 122 is substantially similar to ultrasound device 10 of FIG. 1 with the exception that disposable device 122 is removed from ultrasound transducer 124 following an ultrasound scanning procedure conducted to identify a target area of interest. Following removal of disposable device 122, ultrasound transducer 124 is adhered to the target area of interest via adherable couplant 128 for long-term ultrasound monitoring. FIG. 10 shows ultrasound device 120 having ultrasound transducer 124 and adherable couplant 128, which can be used alone for long-term monitoring, and disposable device 122 having ultrasonic couplant 132, which can be coupled to ultrasound device 120 for ultrasound scanning. Disposable device 122 can be coupled with ultrasound device 120 as illustrated in FIGS. 11C and 11D for ultrasound scanning. Ultrasound device 120 can be separated from disposable device 122 as shown in FIG. 10 following a scanning procedure and attached as a wearable patch to a target area of interest for long-term monitoring.

Disposable device 122 includes frame 130 and ultrasonic couplant 132. Frame 130 is configured to contain ultrasonic couplant 132. Frame 130 and ultrasonic couplant 132 can be substantially similar to frame 22 and ultrasonic couplant 24 of ultrasound device 10 shown in FIG. 1. Ultrasonic couplant 132 can be a solid-like couplant material capable of sliding over the skin of a patient. Preferentially, ultrasonic couplant 132 is formed of a material that can slide over the skin of the patient without leaving a residue. Ultrasonic couplant 132 can be formed of a material having a slippery surface configured to slide over the skin with minimal resistance. Hydrogels can be molded into and will maintain solid shapes and have very low friction over surfaces such as skin. Typical hydrogel coefficients of friction range from0.1 to 0.001. Ultrasonic couplant 132 can have a coefficient of friction of less than 0.1 and, preferably, less than 0.03. Ultrasonic couplant 132 can include, for example, agar-based couplants, water-based couplants, alcohol-based couplants, and/or hydrogels. Frame 130 can extend around a perimeter of ultrasonic couplant 132 such that top and bottom surfaces of ultrasonic couplant 132 are exposed. The top surface of ultrasonic couplant 132 is disposed adjacent to sensor head 126 upon assembly with ultrasound transducer 124. The bottom surface of ultrasonic couplant 132 can protrude outward from a bottom of frame 130 and is configured to interface with skin of the patient during a scanning procedure. The portion of ultrasonic couplant 132 configured to interface with the skin of the patient can have a planar surface with rounded edges, or a convex spherical or curved surface to promote the sliding motion of ultrasound device 120 over the skin during scanning. Unlike liquids, ultrasonic couplant 132 does not deform substantially under external forces, however, ultrasonic couplant 132 can be a flexible material capable of substantially conforming to a surface of the skin to displace air and minimize air pockets between the skin and ultrasonic couplant 132. In some examples, a wetting agent (e.g., water) can be used with ultrasonic couplant 132 to promote the sliding motion and/or to displace air between the skin and ultrasonic couplant 132. Preferably, wetting agents include materials that do not leave a residue on the skin or do not require cleaning of the skin for removal.

Ultrasonic couplant 132 can protrude from a bottom surface of frame 130 as shown in FIGS. 11C and 11D. A thickness by which ultrasonic couplant 132 protrudes from the bottom of frame 130 can be selected to allow ultrasonic couplant 132 to interface with the skin during the scanning procedure and to allow ultrasound device 120 and disposable device 122 to move across the skin of the patient without interference by frame 130. A portion of ultrasonic couplant 132 extending into frame 130 can be a minimal thickness allowing for retention. Preferably, a total thickness of ultrasonic couplant 132 is a minimal thickness that provides for effective movement of ultrasound device 120 and disposable device 122 across the skin and retention of ultrasonic couplant 132 in frame 130.

As illustrated in FIGS. 10 and 11A-11D, frame 130 can have a substantially rectangular shape with tapered side walls to retain ultrasonic couplant 132. Frame 130 is not limited to the configuration illustrated. Other frame shapes and configurations are contemplated. Ultrasonic couplant 132 can be fully or partially recessed in frame 130 such that at least some side walls of frame 130 extend above a top surface of ultrasonic couplant 132 as shown, for example, in FIG. 10. In some examples, three adjoining side walls of frame 130 can extend above a top surface of ultrasonic couplant 132. An inner perimeter of frame 130 at the top surface of ultrasonic couplant 132 can be sized to accommodate sensor head 126 of ultrasound transducer 124.

As shown in FIG. 11C, sensor head 126 can be seated in frame 130 such that side walls of frame 130 overlap side walls (or flange 142) of sensor head 126. Frame 130 can include ejection element 134 configured to release sensor head 126 of ultrasound transducer 124 from frame 130 upon completion of the scanning procedure. Ejection element 134 can be a lever. Ejection element 134 can also be used to retain sensor head 126 in frame 130 during the scanning procedure. Ejection clement 134 can extend upward from a front side wall of frame 130 (opposite a cable of ultrasound transducer 124). Ejection element 134 can include front clip 136 and ejection tab 138. Front clip 136 can extend across ejection element 134 adjacent to a side wall of frame 130. Front clip 136 can protrude outward from ejection element 134 along the inner perimeter of frame 130 to secure a portion of sensor head 126 when seated in frame 130. Front clip 136 can protrude toward an opposite back side wall of frame 130. Front clip 136 can be a ridge configured to restrain upward movement of sensor head 126 when sensor head 126 is seated in frame 130. Ejection tab 138 can extend from a top end of ejection element 134 away from frame 130. Ejection tab 138 can be configured to be pressed by an operator's finger during use to eject frame 130 from sensor head 126. For example, pressing ejection tab 138 can cause front clip 136 to move away from sensor head 126 thereby releasing sensor head 126 and allowing a user to lift sensor head 126 from frame 130. Frame 130 can additionally include back clip 140 configured to retain sensor head 126 during the scanning procedure. Back clip 140 can extend from a back side wall of frame 130 opposite ejection element 134. Back clip 140 can protrude toward the front side wall of frame 130 and ejection element 134. Back clip 140 can be a ridge configured to restrain upward movement of sensor head 126 when sensor head 126 is seated in frame 130. Back clip 140 can serve additional purposes as discussed further herein.

Ultrasound transducer 124 can be substantially similar to ultrasound transducer 12 of ultrasound device 10. Sensor head 126 of ultrasound transducer 124 can be configured to be received and retained in frame 130 for use in an ultrasonic scanning procedure. In some examples, sensor head 126 can include flange 142, which can be retained by attachment mechanisms of frame 130, such as ejection element 134 and back clip 140. Flange 142 can extend outward from side walls of sensor head 126 around a perimeter of sensor head 126. Flange 142 can form a portion of sensor face 144 at a bottom surface of sensor head 126. Sensor face 144 faces ultrasonic couplant 132 of frame 130 when assembled with frame 130. Upon assembly with frame 130, flange 142 can be captured by ejection element 134 disposed at a front end of sensor head 126 and by back clip 140 disposed at an opposite back end of sensor head 126 adjacent to a portion of sensor head 126 to which a cable (not shown) is coupled. During assembly, sensor head 126 can be pressed into frame 130 with a snap fit such that front clip 136 and back clip 140 are disposed on a top surface of flange 142 and inhibit upward movement of sensor head 126 relative to frame 130. During disassembly, ejection tab 138 can be pressed to release front clip 136 allowing sensor head 126 to be lifted away from frame 130 or frame 130 to be lowered from sensor head 126.

Retention and ejection mechanisms including front clip 136, back clip 140, flange 142, and ejection tab 138 are examples of retention and ejection mechanisms that can be used to secure and release an ultrasound transducer from a disposable device for ultrasound scanning and long-term monitoring procedures. Retention and ejection mechanisms can take other forms in other examples and are not limited to the mechanisms illustrated. Furthermore, any of the retention and ejection mechanism disclosed herein can be used in alternative combinations or adapted for use with other ultrasound devices and disposable devices disclosed.

FIGS. 11A-11D show ultrasound device 120 in varying states of assembly for use in an ultrasound scanning procedure. FIG. 11A shows ultrasound transducer 124 positioned above disposable device 122 and adherable couplant 128 prior to assembly. FIG. 11B is a partially exploded view of FIG. 11A, illustrating the disposable components of the devices (disposable device 122 and adherable couplant 128) and the reusable component of the device (ultrasound transducer 124). FIGS. 11C and 11D show ultrasound transducer 124 assembled with disposable device 122 and adherable couplant 128. As described further herein, adherable couplant 128 is configured to transmit ultrasound energy and is used for both ultrasound scanning and long-term monitoring procedures. During ultrasound scanning, adherable couplant 128 is disposed between sensor head 126 and ultrasonic couplant 132 in frame 130 and provides an interface between sensor face 144 and ultrasonic couplant 132. During long-term monitoring, adherable couplant 128 is disposed on sensor face 144 and provides an interface between sensor face 144 and a patient's skin to which ultrasound device 120 is affixed.

Ultrasound transducer 124 is reusable and can be stored separately from adherable couplant 128 and disposable device 122. In some examples, disposable device 122 can be packaged and stored with adherable couplant 128. As shown in FIG. 11A, adherable couplant 128 can be disposed on ultrasonic couplant 132. Specifically, adherable couplant 128 can be disposed on film liner 146. Film liner 146 can be disposed on a top surface of ultrasonic couplant 132 to improve release of adherable couplant 128 from ultrasonic couplant 132 upon detachment of disposable device 122 from ultrasonic device 120. Film liner 146 is formed of a material capable of transmitting ultrasound energy and separating from adherable couplant 128 upon removal of sensor head 126 from frame 130. Film liner 146 can include opening 148. Opening 148 can be a hole positioned on a tab of film liner 146 extending from an end of adherable couplant 128. The position of opening 148 can correspond to the location of back clip 140 on frame 130. Back clip 140 can be received in opening 148. In some examples, back clip 140 can be configured to retain film liner 146 upon removal of sensor head 126 from frame 130. In some examples, film liner 146 can be eliminated and adherable couplant 128 can interface directly with ultrasonic couplant 132.

Upon assembly, sensor head 126 of ultrasound transducer 124 can be seated in frame 130, which can be pre-assembled with adherable couplant 128. Sensor head 126 can be received and retained in frame 130, for example, with a snap fit as previously described. Sensor face 144 can adhere to adherable couplant 128. Adherable couplant 128 is formed of a material capable of transmitting ultrasound energy. Adherable couplant 128 can be formed of a material capable of adhering to skin of a patient. For example, adherable couplant 128 can be an adhesive hydrogel sheet, including but not limited to a high-performance hydrogel such as ST-gel® suitable for medical use. The adhesive hydrogel can include a reinforcing scrim (e.g., adhesive hydrogel can be integrated into a nonwoven fabric). As described further herein, in other examples, adherable couplant 128 can include a plurality of layers capable of transmitting ultrasound energy. Adherable couplant 128 can include, for example, a carrier material with an adhesive material disposed on one side of the carrier material capable of adhering to sensor face 144 and an adhesive material disposed on an opposite side of the carrier material capable of adhering to skin of a patient for long-term monitoring. Sensor face 144 can be tightly secured to frame 130 such that contact between sensor face 144, adherable couplant 128, film liner 146, and ultrasonic couplant 132 displaces air and allows ultrasonic energy to be effectively transmitted therebetween. In some examples, adherable couplant 128 can be formed of a material capable of being removed from ultrasonic couplant 132 upon detachment of disposable device 122 from ultrasound device 120 such that film liner 146 can be eliminated.

When assembled with disposable device 122, as shown in FIGS. 11C and 11D, ultrasound device 10 can be used to scan a patient to identify a target area of interest. As previously disclosed, ultrasonic couplant 132 can be slid across the skin of a patient. In some examples, an additional couplant or wetting agent can be applied to the skin or ultrasonic couplant 132 to facilitate scanning. Once the target area of interest is identified, sensor head 126 can be released from frame 130, for example, by pressing ejection tab 138 and lifting sensor head 126 away from frame 130 or lowering frame 130 from sensor head 126. As sensor head 126 is removed from frame 130, adherable couplant 128, which is adhered to sensor face 144, can be removed from film liner 146 (or ultrasonic couplant 132), leaving ultrasound device 120 as shown in FIG. 10. Film liner 146, if used, would remain on ultrasonic couplant 132 upon removing sensor head 126 from frame 130. Ultrasound device 120 can be secured to the skin of the patient (via adherable couplant 128) at the target area of interest as a wearable patch for long-term ultrasound monitoring. In some examples, an additional anchoring mechanism, such as an adhesive patch, can be used to secure ultrasound device 120 to the skin. For example, an adhesive patch can be applied over sensor head 126 and can be affixed to the skin surrounding sensor head 126.

In some examples, ultrasound device 120 can further include one or more protective barriers (not shown) similar to protective barriers 74 and 76 shown in FIG. 4 and described with respect thereto. For example, a protective barrier can be attached to adherable couplant 128 and can extend to cover sensor head 126 and at least a portion of a cable of ultrasound transducer 124. In some examples, the protective barrier can form a portion of adherable couplant 128 along sensor face 144 such that the protective barrier engulfs sensor head 126. In other examples, the protective barrier can be attached to a portion of sensor face 144 adjacent to adherable couplant 128 (e.g., along an outer perimeter of sensor face 144) and can extend to cover sensor head 126 and a portion of the cable. The protective barrier(s) can protect ultrasound transducer 124 from biological contamination during use. The protective barrier(s) can be disposed of following use of ultrasound device 120. Ultrasound transducer 124 can be reused in subsequent applications with new disposable devices 122 and adherable couplant 128, which may include one or more protective barriers.

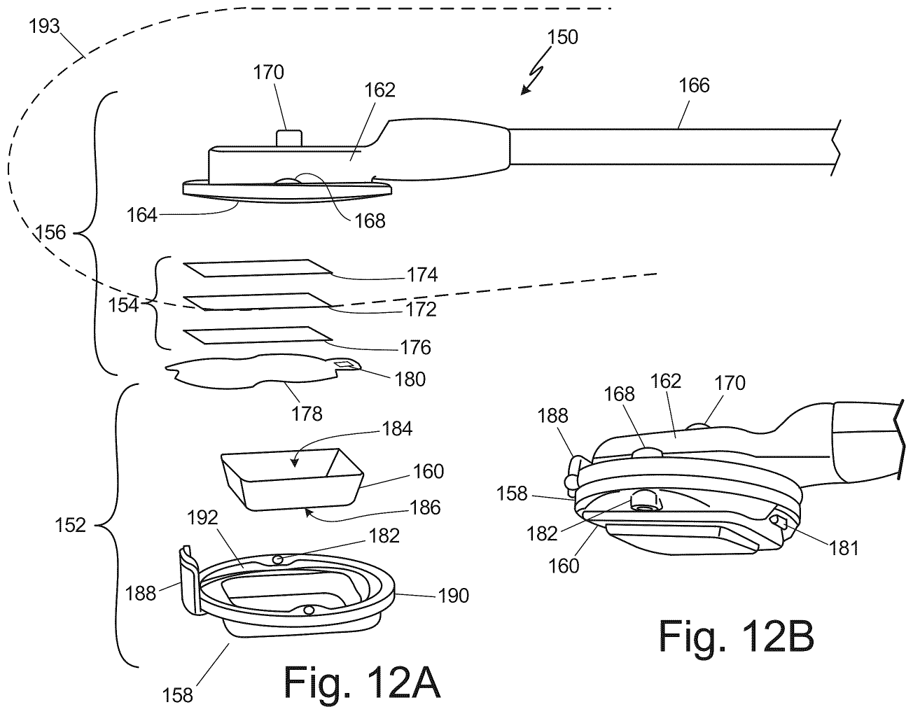

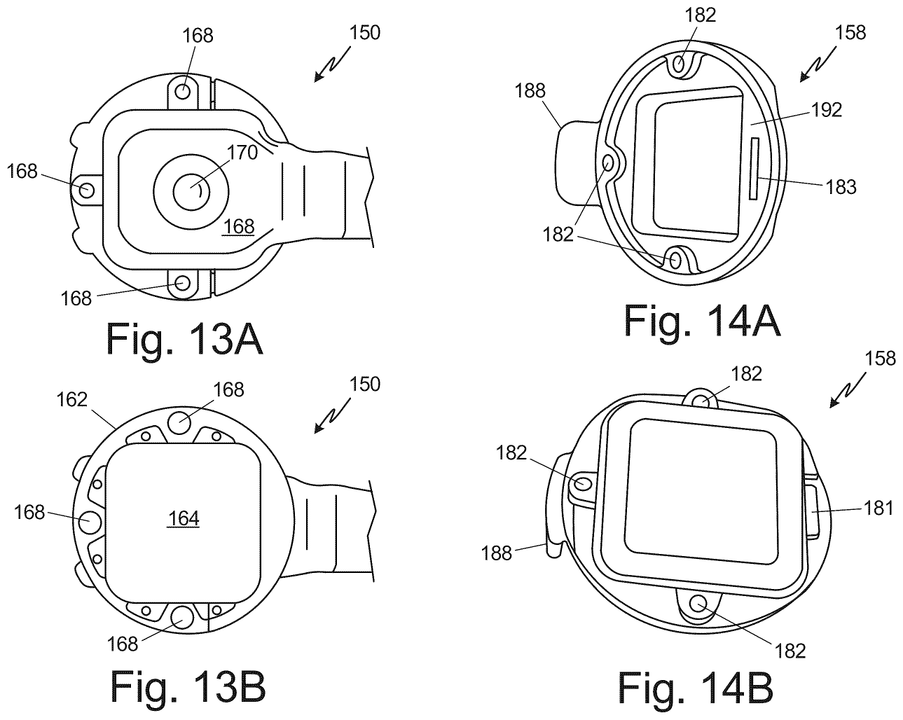

FIGS. 12A and 12B show yet another dual-function ultrasound device. FIG. 12A shows an exploded view of the dual-function ultrasound device. FIG. 12B shows an assembled view of the ultrasound device configured for ultrasound scanning. FIGS. 12A and 12B are discussed together. FIG. 12A shows ultrasound transducer 150, disposable device 152, and adherable couplant 154. Together, ultrasound transducer 150 and adherable couplant 154 form ultrasound device 156, configured for long-term monitoring or configured for use with disposable device 152, as shown in FIG. 12B, for ultrasound scanning. Disposable device 152 includes frame 158 and ultrasonic couplant 160. Ultrasound transducer 150, adherable couplant 154, disposable device 152, frame 158, ultrasonic couplant 160, sensor head 162, sensor face 164, cable 166, sensor attachment mechanism 168, affixing element 170, carrier layer 172, first adhesive layer 174, second adhesive layer 176, film liner 178, opening 180, retention clip 181, frame attachment mechanism 182, slot 183, top surface 184, bottom surface 186, ejection element 188, flange 190, and ledge 192 are shown. FIGS. 12A and 12B show a side view of the dual function ultrasound device. Additional details of ultrasound transducer 150, including sensor head 162, sensor face 164, sensor attachment mechanism 168, and affixing clement 170 are shown in FIGS. 13A and 13B, which show top and bottom views of ultrasound transducer 150, respectively. Additional details of frame 158, including frame attachment mechanism 182, ejection element 188, flange 190, and ledge 192 are shown in FIGS. 14A and 14B, which show top and bottom views of frame 158, respectively.

Ultrasound device 156 is a dual function ultrasound device configured for use with disposable device 152 for ultrasound scanning to identify a target area of interest and configured for use alone (without disposable device 152) as an ultrasound patch for long-term ultrasound monitoring. Ultrasound device 156 is substantially similar to ultrasound device 120 of FIG. 10 and disposable device 152 is substantially similar to disposable device 122 of FIG. 10. A primary distinction between ultrasound device 156 and ultrasound device 120 and between disposable device 152 and disposable device 122 is how ultrasound devices 156 and 120 are attached to disposable devices 152 and 122, respectively. As described further herein, ultrasound device 156 can be secured to disposable device 152 by magnetic attachment.

Ultrasound device 156 includes ultrasound transducer 150 and adherable couplant 154. Ultrasound transducer 150 includes sensor head 162 with sensor face 164, cable 166, and attachment mechanisms 168. Sensor head 162 can additionally include affixing element 170. Ultrasound transducer 150 is substantially similar to ultrasound transducer 12 of ultrasound device 10 and ultrasound transducer 124 of ultrasound device 120. Cable 166 is coupled to sensor head 162 and can extend from a side wall of sensor head 162 adjacent to sensor face 164. Cable 166 can be oriented parallel to and offset from sensor face 164 as disclosed with respect to ultrasound device 10. Sensor face 164 is disposed on a bottom surface of sensor head 162 and configured to interface with adherable couplant 154. Sensor face 164 can include attachment mechanism 168 configured to secure sensor head 162 to frame 158. As shown in FIGS. 13A and 13B, attachment mechanism 168 can be disposed along a perimeter of sensor face 164. Attachment mechanism 168 can include, for example, a plurality of magnets spaced along the perimeter of sensor face 164 adjacent to an outer edge of sensor face 164 or a magnetic ring disposed along a perimeter of sensor face 164 adjacent to an outer edge of sensor face 164. Attachment mechanism 168 can be spaced apart from adherable couplant 154 upon assembly such that attachment mechanism 168 does not interfere with adherable couplant 154. Affixing element 170 can be disposed on a top surface of sensor head 162 opposite sensor face 164. Affixing clement 170 can be configured to secure an adhesive patch to sensor head 162 as described further herein.

Adherable couplant 154 is formed of materials capable of transmitting ultrasound energy between sensor head 162 and ultrasonic couplant 160 during an ultrasonic scanning procedure and between sensor head 162 and the patient's skin during long-term monitoring. Adherable couplant 154 can be formed of a plurality of layers arranged in a stacked configuration and capable of transmitting ultrasound energy. Adherable couplant 154 is configured to affix sensor face 164 to a patient's skin. In some examples, adherable couplant 154 can be formed of multiple materials having different material characteristics suited for adhering to the different materials (sensor face 164 and skin). For example, adherable couplant 154 can include carrier layer 172. Carrier layer 172 can be formed of a material having substantially similar stretchable properties to that of skin. Carrier layer 172 can be, for example, a thermoplastic elastomer (TPE). Carrier layer 172 can be disposed between first adhesive layer 174 and second adhesive layer 176. First adhesive layer 174 is configured to affix carrier layer 172 to sensor face 164. First adhesive layer 174 can be, for example, an acrylic adhesive configured to secure carrier layer 172 to sensor face 164 and maintain adhesion of carrier layer 172 to sensor face 164 when sensor head 162 is detached from frame 158. Second adhesive layer 176 is configured to affix carrier layer 172 skin of patient for long-term monitoring. Second adhesive layer 176 can be, for example, a silicone adhesive configured to conform to a surface of the skin to provide an effective bond for transmitting ultrasound energy over an extended period of time. Second adhesive layer 176 can be selected from a variety of medical grade adhesives suitable for skin adhesion and increased wear time and that have substantial transparency to ultrasound energy.

In other examples, adherable couplant 154 can be a single layer material capable of transmitting ultrasound energy and adhering to skin of a patient. For example, adherable couplant 154 can be an adhesive hydrogel sheet, including but not limited to a high-performance hydrogel such as ST-gel® suitable for medical use. The adhesive hydrogel can include a reinforcing scrim (e.g., adhesive hydrogel can be integrated into a nonwoven fabric).

Adherable couplant 154 (specifically, second adhesive layer 176) can be disposed on film liner 178 when ultrasonic device 156 is attached to disposable device 152. Film liner 178 can be substantially similar to film liner 146 shown in FIGS. 11A and 11B. Film liner 178 is disposed between adherable couplant 154 and ultrasonic couplant 160. As disclosed with respect to ultrasonic device 120, film liner 178 can be packaged with adherable couplant 154. Adherable couplant 154, including film liner 178, can be packaged with disposable device 152 or packaged separately from disposable device 152 and assembled with disposable device 152 prior to use. Film liner 178 is formed of a material capable of transmitting ultrasonic energy and separating from adherable couplant 154 upon removal of sensor head 162 from frame 158. Film liner 178 covers the skin facing side of the adhesive couplant 154 (e.g., second adhesive layer 176 shown in FIG. 12 or the skin facing side of a single-layer adherable couplant). Film liner 178 can include opening 180. Opening 180 can be substantially similar to opening 148 of film liner 146 shown in FIGS. 11A and 11B. Opening 180 can be received in and retained by retention clip 181 (FIG. 12B) in frame 158. Retention clip 181 can be configured to retain film liner 178 in disposable device 152 when sensor head 162 is detached from frame 158 and adherable couplant 154, adhered to sensor face 164, is pulled away from film liner 178. Retention clip 181 can be disposed on a bottom side of frame 158. Opening 180 can be formed through a tab of film liner 178, which can be received through slot 183 on a top side of frame 158 and attached to retention clip 181. Film liner 178 can be shaped to fit in frame 158. For example, film liner 178 can include additional holes and/or cutouts configured to receive attachment mechanism 182 or other features on a surface of frame 158 configured to receive sensor head 162. Film liner 178 can cover and extend outward from ultrasonic couplant 160 in frame 158 such that film liner 178 covers a portion of a surface of frame 158. In some examples, adherable couplant 154 can be formed of a material capable of being removed from ultrasonic couplant 160 upon detachment of disposable device 152 from ultrasound device 156 such that film liner 178 can be eliminated.

Disposable device 152 includes frame 158 and ultrasonic couplant 160. Frame 158 and ultrasonic couplant 160 can be substantially similar to frame 130 and ultrasonic couplant 132 of disposable device 122. Frame 158 is configured to contain ultrasonic couplant 160. Ultrasonic couplant 160 can be a solid-like couplant material capable of sliding over skin of a patient. Preferentially, ultrasonic couplant 160 is formed of a material that can slide over skin without leaving a residue. Ultrasonic couplant 160 can be formed of a material having a slippery surface configured to slide over the skin with minimal resistance. Ultrasonic couplant 160 can include, for example, agar-based couplants, water-based couplants, alcohol-based couplants, and/or hydrogels. Hydrogels can be molded into and will maintain solid shapes and have very low friction over surfaces such as skin. Typical hydrogel coefficients of friction are 0.1 to 0.001. Ultrasonic couplant 160 can have a coefficient of friction of less than 0.1 and, preferably, less than 0.03. Frame 158 can extend around a perimeter of ultrasonic couplant 160 such that top and bottom surfaces 184, 186 of ultrasonic couplant 160 are exposed. Top surface 184 of ultrasonic couplant 160 is disposed adjacent to sensor head 162 upon assembly with ultrasound transducer 150. Bottom surface 186 of ultrasonic couplant 160 can protrude outward from a bottom of frame 158 and is configured to interface with skin of the patient during a scanning procedure. Bottom surface 186 of ultrasonic couplant 160 can have a planar surface with rounded edges, or a convex spherical or curved surface to promote the sliding motion of ultrasound device 156 and disposable device 152 over the skin during scanning. Unlike liquids, ultrasonic couplant 160 does not deform substantially under external forces, however, ultrasonic couplant 160 can be a flexible material capable of substantially conforming to a surface of the skin to displace air and minimize air pockets between the skin and ultrasonic couplant 160. In some examples, a wetting agent (e.g., water) can be used with ultrasonic couplant 160 to promote the sliding motion and/or to displace air between the skin and ultrasonic couplant 160. Preferably, wetting agents include materials that do not leave a residue on the skin or do not require cleaning of the skin for removal.

Ultrasonic couplant 160 can have a thickness suitable for transmitting ultrasound energy received from ultrasound transducer 150 and from a target location in a patient's body. Ultrasonic couplant 160 can protrude from a bottom surface of frame 158 as described with respect to disposable device 122 shown in FIGS. 11C and 11D. A thickness by which ultrasonic couplant 160 protrudes from the bottom of frame 158 can be selected to allow ultrasonic couplant 160 to interface with the skin during the scanning procedure and to allow ultrasound device 156 to move across the skin of the patient without interference by frame 158.

As illustrated in FIGS. 12A and 12B, ultrasonic couplant 160 can have a rectangular shape with side walls that taper inward from top surface 184 to bottom surface 186. Side walls of frame 158 can have a tapered shape corresponding to the shape of ultrasonic couplant 160, such that an opening in frame 158 through which bottom surface 186 of ultrasonic couplant 160 protrudes is smaller than top surface 184 of ultrasonic couplant 160. Ultrasonic couplant 160 is thereby retained in frame 158. The opening in frame 158 can be larger than bottom surface 186 of ultrasonic couplant 160 thereby allowing a portion of ultrasonic couplant 160 including bottom surface 186 to protrude from frame 158.

Frame 158 can include flange 190. As illustrated in FIGS. 12A and 14A, flange 190 can be disposed at a top surface of frame 158 and can extend outward in a plane parallel to a top surface of ultrasonic couplant 160. Flange 190 can include attachment mechanism 182 and ejection element 188. In some examples, flange 190 can include ledge 192. Ledge 192 can be recessed from the top surface of frame 158 and can form an inner perimeter of flange 190. Ledge 192 can include the one or more attachment mechanisms 182. Ledge 192 can be configured to interface with sensor face 164 of sensor head 162, which can have a corresponding protruding shape (not shown). Ultrasonic couplant 160 can be disposed in frame 158 such that top surface 184 of ultrasonic couplant 160 is disposed in a common plane with ledge 192. Film liner 178 can be disposed on ultrasonic couplant 160 and ledge 192. Sensor head 162, when assembled with frame 158 can press against ultrasonic couplant 160. Sensor face 164 can be received on ledge 192 and retained by attachment mechanisms 168 and 182.

Attachment mechanism 182 (shown in FIGS. 14A and 14B) can include one or more magnets configured to attract and retain one or more magnets (attachment mechanism 168 shown in FIGS. 13A and 13B) on sensor head 162. In some examples, attachment mechanism 182 or attachment mechanism 168 can include a plurality of magnetic posts and the other of attachment mechanism 182 and attachment mechanism 168 can include a plurality of corresponding holes with magnetic features configured to receive the plurality of magnetic posts. In another example, frame 158 can be magnetic and configured to attract and retain attachment mechanism 168 on sensor head 162, which can include one or more magnets.

Ejection element 188 can extend upward from frame 158 at a front side of frame 158. The front side of frame 158 corresponds to a front side of sensor head 162 disposed opposite cable 166. Ejection element 188 is configured to facilitate disassembly of ultrasound device 156 from disposable device 152. For example, ejection element 188 can be configured to be pressed by a user's finger to help break the magnetic attachment between sensor head 162 and frame 158. In some examples, ejection element 188 can include a clip portion similar to front clip 136 of frame 130 shown in FIG. 11A and described with respect thereto. The clip portion can be configured to provide an additional retention feature to secure sensor head 162 to frame 158 for use during the scanning procedure. In such example, pressing ejection element 188 forward can additionally release sensor head 162 from the clip portion when scanning is complete. Additionally, ejection element 188 can help align ultrasound transducer 150 at the time of coupling with disposable device 152. It is important to ensure proper orientation of sensor head 162 in frame 158 as misalignment or misorientation may not be corrected or may not be easily corrected once adherable couplant 154 is affixed to sensor face 164.

Frame 158 is not limited to the configuration illustrated in FIGS. 12A, 12B, 14A, and 14B. Other frame shapes and configurations are contemplated. Retention and ejection mechanisms including attachment mechanism 168 on sensor head 162, attachment mechanism 182 on frame 158, and ejection element 188 are examples of retention and ejection mechanisms that can be used to secure and release an ultrasound transducer from a disposable device for ultrasound scanning and long-term monitoring procedures. Retention and ejection mechanisms can take other forms in other examples.

Ultrasound transducer 150 can be assembled with adherable couplant 154 and disposable device 152 for an ultrasound scanning procedure. Sensor head 162 of ultrasound transducer 150 can be seated in frame 158, which can be pre-assembled with adherable couplant 154 and film liner 178. Sensor head 162 can be secured to frame 158 by attachment mechanisms 168 and 182. Sensor face 164 can be adhered to adherable couplant 154 by first adhesive layer 174. Sensor face 164 presses adherable couplant 154 and film liner 178 against ultrasonic couplant 160 such that contact between sensor face 164, adherable couplant 154, film liner 178, and ultrasonic couplant 160 allows ultrasonic energy to be effectively transmitted therebetween. Ultrasound device 156 in combination with disposable device 152 can be used to scan a patient to identify a target area of interest for long-term monitoring. As previously disclosed, ultrasonic couplant 160 can be slid across the skin of a patient. In some examples, an additional couplant or wetting agent can be applied to the skin or ultrasonic couplant 160 to facilitate scanning. Once the target area of interest is identified, sensor head 162 can be released from frame 158, for example, by pressing ejection element 188. As sensor head 162 is removed from frame 158, adherable couplant 154, which is adhered to sensor face 164, can be removed from film liner 178 (or ultrasonic couplant 160). The resulting ultrasound device 156, including ultrasound transducer 150 and adherable couplant 154, can be secured to the skin of the patient via adherable couplant 154 (i.e., second adhesive layer 174) at the target area of interest for long-term ultrasound monitoring.

In some examples, an additional anchoring mechanism, such as an adhesive patch as described further herein, can be used to secure ultrasound device 156 to the skin. For example, an adhesive patch can be applied over sensor head 162 and can be affixed to the skin surrounding sensor head 162. In some examples, an adhesive patch can include a hole configured to receive affixing element 170. Affixing element 170 can be used to properly locate and/or secure the adhesive patch to sensor head 162.

In some examples, ultrasound device 156 can further include one or more protective barriers 193 (shown in phantom) similar to protective barriers 74 and 76 shown in FIG. 4 and described with respect thereto. For example, protective barrier 193 can be a protective sheath attached to adherable couplant 154 or to sensor face 164 adjacent to adherable couplant 154 and can extend to cover sensor head 162 and at least a portion of cable 166 of ultrasound transducer 150. In some examples, protective barrier 193 can additionally form a portion of adherable couplant 154 along sensor face 164 such that protective barrier 193 engulfs sensor head 162. For example, carrier layer 172 can be a protective barrier that can extend around sensor head 162 as a protective sheath or can attach to a protective sheath. Protective barrier 193 can protect ultrasound transducer 150 from biological contamination during use. In examples where protective barrier 193 forms a portion of the ultrasonic couplant, protective barrier 193 is formed of a material capable of transmitting ultrasonic energy. Protective barrier 193 can be disposed of following use of ultrasound device 156. Ultrasound transducer 150 can be reused in subsequent applications with new disposable devices 152 and adherable couplant 154, which may include one or more protective barriers.

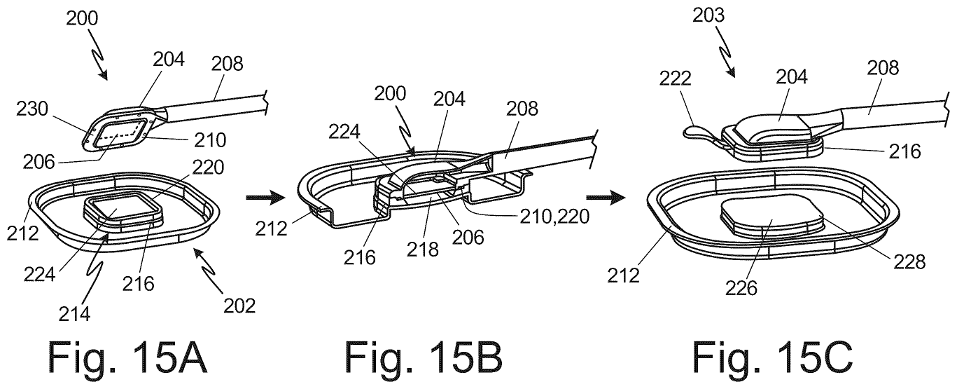

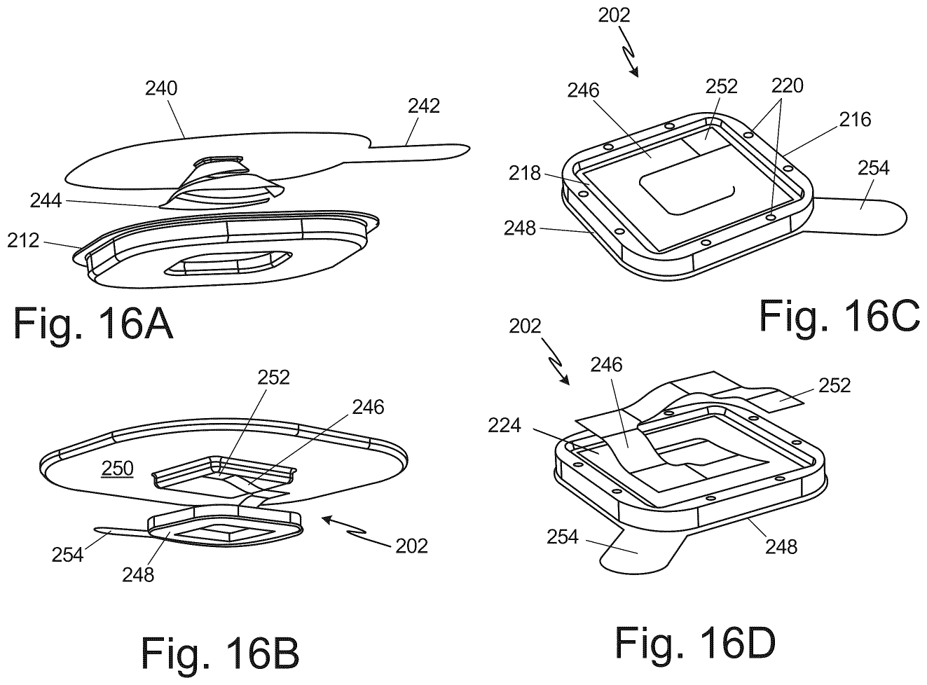

FIGS. 15A-15C are perspective views of a method of assembling a dual-function ultrasound device according to the present disclosure. Ultrasound transducer 200, packaged device 202, and assembled device 203 are shown. Ultrasound transducer includes ultrasound sensor head 204 with sensor face 206, cable 208, and attachment mechanism 210. Packaged device 202 is stored in packaging dish 212. Packaged device 202 includes disposable device 214 with frame 216, ultrasonic couplant 218, attachment mechanism 220, ejection element 222, and adherable couplant 224. Packaging dish 212 can include a lid or cover (shown in FIGS. 16A and 16B) and seal 226.

Ultrasound transducer 200 can be substantially similar to ultrasound transducer 150 shown in FIG. 12 and described with respect thereto. Disposable device 214 can be substantially similar to disposable device 152 shown in FIGS. 12A and 12B and described with respect thereto. Adherable couplant 224 can be substantially similar to adherable couplant 154 shown in FIG. 12A or adherable couplant 128 shown in FIG. 11A and described with respect thereto.

Disposable device 214 and adherable couplant 224 can be prepackaged in packaging dish 212. Adherable couplant 224 can be disposed on ultrasonic couplant 218 provided in frame 216. Adherable couplant 224 can be adhered to a film liner (e.g., film liner 178) on ultrasonic couplant 218 as described with respect to FIG. 12A. Packaging dish 212 can include a lid or cover (shown in FIGS. 16A and 16B), which can cover a top surface of packaged device 202. For example, a lid can extend fully across a top of packaging dish 212 and can be secured to edges of packing dish 212.

The lid can be removed prior to assembling packaged device 202 with ultrasound transducer 200. Removing the lid can expose an adhesive layer of adherable couplant 224 (i.e., first adhesive layer 174 shown in FIG. 12). FIG. 15A illustrates exposed adherable couplant 224 in packaged device 202 following removal of the lid from packaging dish 212.

In a next step, illustrated in FIG. 15B, sensor head 204 of ultrasound transducer 200 can be pressed against adherable couplant 224 and frame 216 of packaged device 202 to form assembled device 203. Packaged device 202 can remain in packaging dish 212 as ultrasound transducer 200 is assembled with packaged device 202. Sensor head 204 can be secured to frame 216 mechanically or magnetically by attachment mechanisms 210 and 220. Attachment mechanisms 210 and 220 can include, for example, one or more magnetic elements or other elements configured for mechanical attachment. Sensor head 204 and frame 216 can be configured with any of the retention features disclosed with respect to FIGS. 2-4, 10, 11A-11D, 12A, and 12B, and variations thereof. As illustrated in FIG. 15A, sensor face 206 can protrude from a bottom surface of sensor head 204 forming recessed rim 230 around sensor face 206. Adherable couplant 224 can be recessed in frame 216. A depth of the recess of adherable couplant 224 can correspond to a depth of recessed rim 230 on sensor face 206 such that sensor face 206 fits tightly against adherable couplant 224 when assembled as illustrated in FIG. 15B.

In some examples, a protective barrier (not shown) can be housed in packaging dish 212. The protective barrier can be coupled, for example, to adherable couplant 224 as described with respect to FIGS. 12A and 12B. The protective barrier can include a protective sheath that can be stored in packaging dish 212 around frame 216. In some examples, packaging dish can include platform 228 on which frame 216 and ultrasonic couplant 218 are seated. Platform 228 can be provided to increase a depth of packaging dish 212, which can provide additional storage space for the protective sheath. The protective sheath can be unwrapped and pulled back to cover sensor head 204 and at least a portion of cable 208.

Following assembly, assembled device 203 can be lifted from packaging dish 212 as illustrated in FIG. 15C. Ultrasonic couplant 218 can be released from seal 226 in packaging dish 212, leaving a bottom surface of ultrasonic couplant 218 extending from frame 216 exposed and ready for use. In other examples, a seal can remain on ultrasonic couplant 218 upon removal of assembled device 203 from packaging dish 212 as illustrated in FIGS. 16B-16D (seal 248). The seal can be removed prior to use of assembled device 203. Packaging dish 212 can be disposed of. Assembled device 203 can be used in an ultrasound scanning procedure on a patient's body to identify a target area of interest as described, for example, with respect to the devices illustrated in FIGS. 11C and 11D, and FIGS. 12A and 12B. Once the target area of interest is identified, disposable device 214 can be removed, for example, by pressing ejection element 222. Disposable device 214 can be disposed of. Adherable couplant 224 can remain adhered to sensor head 204 upon removal of disposable device 214 and can be affixed to the target area of interest for long-term monitoring as previously described. Upon completion of long-term monitoring, adherable couplant 224 can be removed from ultrasound transducer 200. Ultrasound transducer 200 can be coupled to a new packaged device, as illustrated in 15A, in a subsequent procedure.

FIG. 16A is a perspective view of packaging for packaged device 202 of FIG. 15A during removal of a packaging lid. FIG. 16B is a perspective view of another example of packaging for packaged device 202 of FIG. 15A during removal of a packaging lid. FIG. 14C is a perspective top view of packaged device 202 of FIG. 16B with a protective film layer. FIG. 16D is a perspective top view of packaged device 202 of FIG. 16B during removal of the protective film layer. FIGS. 16A-16D are discussed together. Packaging dish 212, lid 240, tab 242, and seal 244 are shown in FIG. 16A. FIG. 16B shows packaged device 202, seals 246 and 248, and lid 250, which can be provided as an alternative to lid 240 in FIG. 16A. Frame 216, ultrasonic couplant 218, attachment mechanism 220, and tabs 252 and 254 are shown in FIG. 16C. Adherable couplant 224 is shown in FIG. 16D.

FIG. 16A shows lid 240 being removed from packaging dish 212 of FIG. 15A. Lid 240 can be a protective cover having tab 242, which can be used to pull lid 240 away from packaging dish 212. Seal 244 can be attached to lid 240 and adherable couplant 224. Seal 244 can be a release liner or backing paper as known in the art configured to be removed cleanly (without residue) from adherable couplant 224. Seal 244 can be cut in a pattern to facilitate removal. For example, a tab of seal 244 (e.g., similar to tab 252 of seal 246 shown in FIGS. 16B-16D) can be attached to lid 240, which can pull seal 244 from adherable couplant 224 in a pattern, such as a spiral or raster pattern.

FIG. 16B shows removal of lid 250 from packaged device 202. Lid 250 is an alternative to lid 240 of FIG. 16A. Lid 250 can be configured for use with packaging dish 212 or similar packaging dish. FIG. 16B shows seal 246 being pulled from packaged device 202 and, specifically, from adherable couplant 224. Seal 246 can be substantially the same as seal 244 described with respect to FIG. 16A. Seal 246 can include a patterned cut and tab 252 provided for case of removal as previously described. Tab 252 can be attached to lid 250. When lid 250 is removed, lid 250 can pull tab 252 and subsequently, the remainder of seal 246 in a spiral or other pattern.

Seal 248 is disposed on ultrasonic couplant 218 opposite seal 246 on packaged device 202. Seal 248 can extend across the surface of ultrasonic couplant 218 and frame 216. Seal 248 can include tab 254 provided for case of removal of seal 248 prior to use of assembled ultrasound device 203.

FIGS. 16C and 16D show a bottom side of packaged device 202 with seals 246 and 248 and tabs 252 and 254. Seal 246 is disposed on adherable couplant 224 and can be cut in a pattern for case of removal as previously described. Seal 246 includes tab 252, which can be attached to lid 240 or lid 250. Seal 246 can be removed from adherable couplant 224 in a spiral pattern as illustrated in FIG. 16D by pulling tab 252. Seal 248 is disposed on ultrasonic couplant 218. Seal 248 can be removed from ultrasonic couplant 218 by pulling tab 254. FIGS. 16B-16D are simplified drawings of packaged device 202 and do not include ejection element 222 as shown in FIG. 15C.

It will be understood by one of ordinary skill in the art that the packing and seals shown in FIGS. 15A-15C and 16A-16D can be adapted for use with any of the disposable devices described herein and are not limited to the configurations shown.

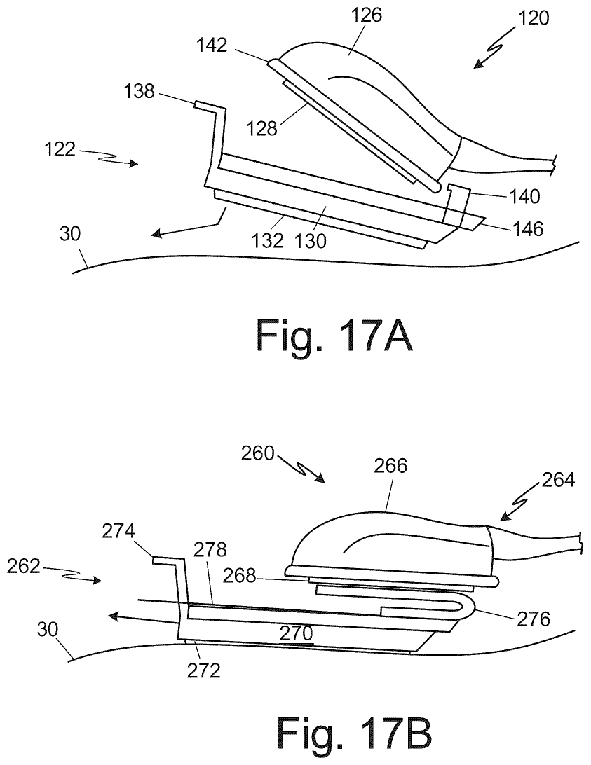

FIGS. 17A and 17B illustrate alternative methods of detaching a disposable device used for ultrasound scanning from an ultrasound device used for long-term monitoring and subsequent attachment of the ultrasound device to the skin of the patient for long-term monitoring. While particular examples are illustrated, it will be understood by one of ordinary skill in the art that any of the disclosed ultrasound devices and disposable devices can be adapted to accommodate the disclosed detachment methods.

FIG. 17A shows a cross-sectional view of the dual-function ultrasonic device of FIG. 10 during removal of disposable device 122 used for ultrasound scanning from ultrasound device 120 used for long-term monitoring. FIG. 15A shows ultrasound device 120 having ultrasound transducer 124, sensor head 126, adherable couplant 128, and flange 142; disposable device 122, having frame 130, ultrasonic couplant 132, ejection tab 138, back clip 140, and film liner 146; and skin 30. Following an ultrasound scanning procedure, disposable device 122 can be detached or ejected from ultrasound device 120 and discarded. While positioned above the target area of interest, ejection tab 138 can be pressed forward or away from flange 142 of sensor head 126 to release the front end of sensor head 126 from frame 130. A user can then lift the front end of sensor head 126 away from disposable device 122 and/or lower a front end of disposable device 122 away from sensor head 126. As the front end of sensor head 126 is released from disposable device 122, a back end of sensor head 126 (at flange 142) can remain engaged with back clip 140 in a hinged connection. Film liner 146 can be secured to back clip 140 as previously described and can separate from adherable couplant 128 upon removal of sensor head 126 from frame 130. Once film liner 146 is fully released from adherable couplant 128, disposable device 122 can be pulled out from under ultrasound device 120 while ultrasound device 120 remains above the target area of interest. Ultrasound device 120 can then be lowered and pressed against skin 30 in the target area of interest. An additional adhesive patch can be applied over ultrasound device 120 to secure ultrasound device 120 to skin 30 for long-term monitoring.

FIG. 17B shows a cross-sectional view of an alternative method of detaching a disposable device used for ultrasound scanning from an ultrasound device used for long-term monitoring. FIG. 17B shows ultrasound device 260 and disposable device 262. Ultrasound device 260 includes ultrasound transducer 264, sensor head 266 and adherable couplant 268. Disposable device 262 includes frame 270, ultrasonic couplant 272, ejection element 274, film liner 276, and release strip 278. Ultrasound device 260 can be substantially similar to ultrasound devices 120 and 156 and disposable device 262 can be substantially similar to disposable device 122 and 152 previously described. Ultrasound device 260 and disposable device 262 include modified retention and ejection mechanisms configured to secure ultrasound device 260 to disposable device 262 and remove ultrasound device 260 from disposable device 262. Ultrasound device 260 is configured for use as an ultrasound patch for long-term monitoring. Disposable device 262 is configured for use with ultrasound device 260 for scanning a patient's body to identify a target area of interest prior to long-term monitoring. Ultrasound device 260 can be removed from disposable device 262 by sliding disposable device 262 away from the ultrasound device 260. This method of separation allows a user to maintain a position of ultrasound device 260 directly above the target area of interest once identified. For example, ultrasound device 260 can be assembled with disposable device 262 and scanned across a patient's body to find a target area of interest as previously described. Once the target area of interest is identified, a user can eject disposable device 262 from ultrasound device 260 by sliding disposable device 262 out from under ultrasound device 260 as indicated by the arrows showing a direction of movement of disposable device 262. Ejection element 274 can be pressed away from frame 270 to disengage attachment mechanisms (not shown) and push disposable device 262 away from the target area of interest. Release strip 278 can be attached to frame 270 and to an end of film liner 276 to pull film liner 276 away from adherable couplant 268 as frame 270 is moved away from sensor head 266. For example, release strip 278 can be attached to a front end of frame 270 (e.g., attached to ejection element 274) and a back end of film liner 276 such that release strip 278 extends under or film liner 276 in assembly. Release strip 278 can be similar to release strip 96 shown in FIGS. 8A and 8B and described with respect thereto. Film liner 276 can be transparent to ultrasonic energy. As frame 270 is moved away from sensor head 266, release strip 278 can pull the back end of film liner 276 such that film liner 276 begins to fold and pull away from adherable couplant 268 as shown. Once disposable device 262 is fully removed from ultrasound device 260, ultrasound device 260 can be pressed down on skin 30 and adhered to skin 30 for long-term monitoring.

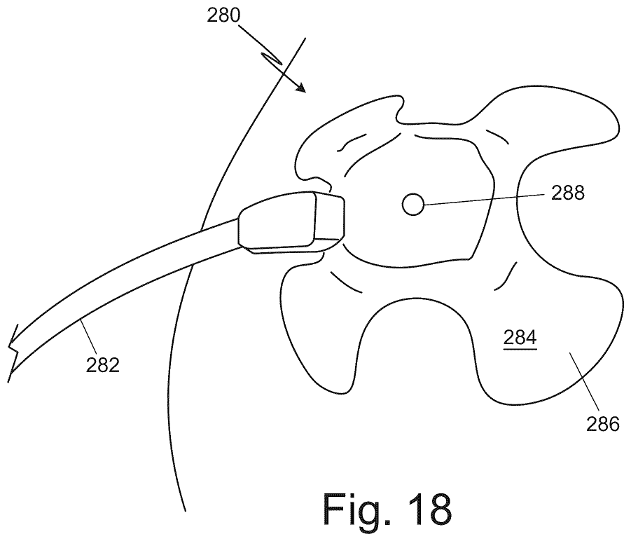

FIG. 18 is a perspective view of an adhesive patch for securing the dual-function ultrasound devices disclosed herein to skin of a patient. FIG. 16 shows ultrasound device 280 with cable 282 and adhesive patch 284 with adhesive strips 286 and locating feature 288. Ultrasound device 280 can be substantially similar to ultrasound devices 120, 156, and 260 disclosed herein and configured for use as a wearable patch for long-term monitoring. Ultrasound device 280 can include a sensor head with an adherable couplant as previously described, which can be adhered to skin of a patient. Adhesive patch 284 can be configured to provide an additional anchoring mechanism for securing ultrasound device 280 to the patient's body.

Adhesive patch 284 can be disposed over a top surface of the sensor head and can be adhered to skin surrounding the sensor head. Locating feature 288 can be used to locate adhesive patch 284 on the sensor head and/or help secure adhesive patch 284 to the sensor head as described with respect to ultrasound device 156 illustrated in FIG. 12. Adhesive patch 284 can include a mechanism similar to release strip 96 shown in FIGS. 8A and 8B and described with respect thereto to remove a cover layer (e.g., release liner or backing paper) from an adhesive surface of adhesive patch 284. In some examples, a string or strip can be attached to a cover layer on adhesive patch 284 and can be configured to remove the cover layer from each of a plurality of adhesive strips 286 of adhesive patch 284 when pulled.

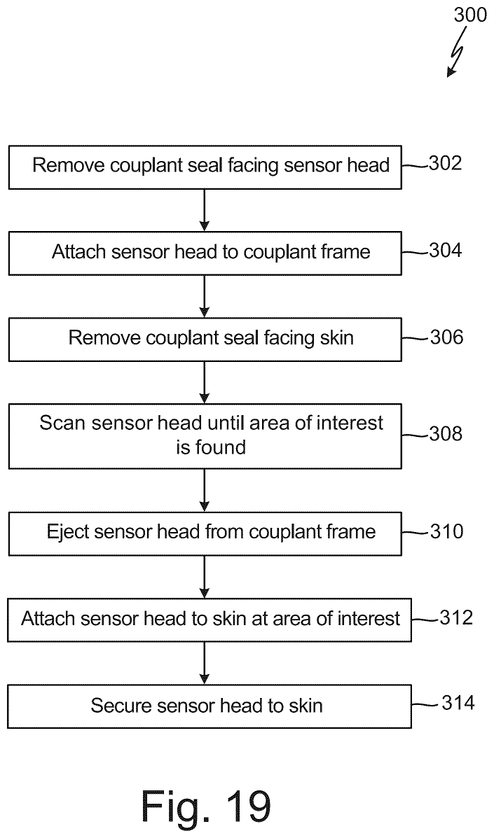

FIG. 19 is a flow chart of a method for assembly and use of the dual-function ultrasound devices of FIGS. 10, 12, and 14A-14C. FIG. 19 shows method 300.

Step 302 includes removing a seal or film liner from a couplant facing a sensor head of an ultrasound transducer. As previously described, a disposable device can be packaged with an adherable couplant configured to be adhered to a face of the sensor head upon assembly. The adherable couplant can be protected with a film liner in the packaging. The film liner can be removed independently or with removal of a lid of the packaging.

Step 304 includes attaching the sensor head to a frame. As previously described, the frame can include an ultrasonic couplant for scanning a patient's body to identify a target area of interest and the adherable couplant disposed on the ultrasonic couplant. The sensor head is attached to the frame in a manner that brings a face of the sensor head in contact with the adherable couplant such that ultrasound energy can be transferred therebetween. The adherable couplant can be configured to adhere to the sensor face. The sensor head can be secured to the frame with retention features previously described.

Step 306 includes removing a seal or film liner from the ultrasonic couplant facing the skin of the patient. As previously described, a portion of the ultrasonic couplant protruding from a bottom of the frame can be protected with a seal or film liner. The film liner can be removed as the assembled ultrasound device is removed from the packaging or can be removed from the ultrasonic couplant prior to use.

Step 308 includes scanning the ultrasound device, including sensor head and disposable device, across skin of the patient until a target area of interest is identified. Step 308 includes transmitting ultrasound energy from the ultrasound transducer and receiving transmitted ultrasound energy from the area scanned as known in the art. In some examples, step 308 can include adding a couplant or wetting agent to the skin and/or the ultrasonic couplant to facilitate the transmission of the ultrasound energy and to facilitate sliding of the ultrasonic couplant on the skin. Preferably, any added couplant or wetting agent is a material that can dry without leaving a residue and does not interfere with adhering the ultrasound device to the skin once the target area of interest is located.

Step 310 includes ejecting the ultrasound device from the disposable device and, specifically, ejecting the sensor head from the frame. As previously described, the assembled device can include an ejection element, such as a lever which can be pushed to release the sensor head from the frame. In some examples, the sensor head can be lifted from the frame, or the frame can be lowered from the sensor head. Alternatively, the frame can be slid away from the sensor head while maintaining a position of the sensor head above the target area of interest. During ejection of the ultrasound device, the adherable couplant is separated from the disposable device. At the conclusion of step 310, one side of the adherable couplant is attached to the sensor face and the other side of the adherable couplant is exposed and ready to be attached to the skin of the patient.

Step 312 includes attaching the ultrasound device to the skin of the patient at the target area of interest. As previously discussed, the ultrasound device includes the ultrasound transducer and adherable couplant. The adherable couplant can be formed of a material capable of adhering to skin for long-term monitoring.

Step 314 includes securing the ultrasound transducer to the skin. As previously disclosed, an adhesive patch can be applied over the sensor head and to the skin surrounding the sensor head to further secure the ultrasound device to the skin for long-term monitoring.

The disclosed dual-function ultrasound devices can be used to scan a patient's body to identify a target area of interest and to affix a reusable ultrasound transducer to a patient's body at the target area of interest for long-term monitoring. The disclosed ultrasound devices are simple to deploy and require no additional devices or cleaning procedures, which is important in busy, time-critical settings such as the operating room and the intensive care unit.

The features of each of the devices, including but not limited to frames, adherable couplant, ultrasonic couplant, sensor heads, retention features, and ejection elements disclosed herein can be used interchangeably or adapted for use with other disclosed embodiments and are not limited to the embodiments for which they are disclosed.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Detailed Embodiments

The following are non-exclusive descriptions of possible embodiments of the present invention.

A dual-function ultrasound device for ultrasonic scanning and use as a wearable ultrasonic patch includes an ultrasound transducer, a detachable frame including an ultrasonic couplant coupled to the ultrasound transducer, and a protective barrier configured to isolate the ultrasound transducer from biological contamination.

The dual-function ultrasound device of the preceding paragraph can optionally include, additionally and/or alternatively, any one or more of the following features, configurations and/or additional components:

In an embodiment of the foregoing dual-function ultrasound device, the ultrasound transducer can include a sensor head configured to be received in a first opening in the detachable frame.

In an embodiment of any of the foregoing dual-function ultrasound devices, the sensor head can include a first attachment mechanism and the detachable frame can include a second attachment mechanism. The first and second attachment mechanisms can be configured to interact to retain the sensor head in the detachable frame.

In an embodiment of any of the foregoing dual-function ultrasound devices, the detachable frame can include a second opening opposite the first opening. The ultrasonic couplant can protrude outward from the second opening.

In an embodiment of any of the foregoing dual-function ultrasound devices, walls of the detachable frame can taper inward toward the second opening.

In an embodiment of any of the foregoing dual-function ultrasound devices, the ultrasonic couplant can be a solid-like couplant capable of sliding over skin for ultrasonic scanning.

In an embodiment of any of the foregoing dual-function ultrasound devices, the ultrasonic couplant can be a hydrogel.

In an embodiment of any of the foregoing dual-function ultrasound devices, the protective barrier can include a sheath coupled to the detachable frame and extending around a sensor head and at least a portion of a cable of the ultrasound transducer.

In an embodiment of any of the foregoing dual-function ultrasound devices, the protective barrier further can include a film disposed in the detachable frame. The film can separate a layer of the ultrasonic couplant from the ultrasound transducer.

In an embodiment of any of the foregoing dual-function ultrasound devices, the detachable frame can include adhesive strips configured to attach the detachable frame to skin of a human body.

In an embodiment of any of the foregoing dual-function ultrasound devices, the adhesive strips can be affixed to a bottom surface of the detachable frame.

In an embodiment of any of the foregoing dual-function ultrasound devices, the ultrasound transducer can further include a sensor head having a sensor face, the sensor face coupled to the ultrasonic couplant; and a cable extending from the sensor head parallel to the sensor face.

In an embodiment of any of the foregoing dual-function ultrasound devices, the ultrasound transducer can have a thin profile with a height to width ratio of less than half.

A dual-function ultrasound device for ultrasonic scanning and use as a wearable ultrasonic patch includes an ultrasound transducer, an adherable couplant attached to the ultrasound transducer, and a frame containing an ultrasonic couplant. The frame is detachably coupled to the ultrasound transducer via the adherable couplant.

The dual-function ultrasound device of the preceding paragraph can optionally include, additionally and/or alternatively, any one or more of the following features, configurations and/or additional components:

In an embodiment of the foregoing dual-function ultrasound device, the adherable couplant can include a material configured to adhere the ultrasound transducer to skin of a human body.

In an embodiment of any of the foregoing dual-function ultrasound devices, the adherable couplant can include an adhesive hydrogel capable of transmitting ultrasonic energy.

In an embodiment of any of the foregoing dual-function ultrasound devices, the adherable couplant can include a reinforcing scrim capable of transmitting ultrasonic energy

In an embodiment of any of the foregoing dual-function ultrasound devices, the adherable couplant can include first and second adhesive layers and a film liner. The first adhesive layer can be configured to adhere the adherable couplant to a face of the ultrasound transducer. The second adhesive layer can be configured to adhere the ultrasound transducer to the skin. The film liner can be disposed between the first and second adhesive layers. Each of the first and second adhesive layers and the film liner can be capable of transmitting ultrasonic energy.

In an embodiment of any of the foregoing dual-function ultrasound devices, wherein the adherable couplant can include a thermoplastic elastomer.

In an embodiment of any of the foregoing dual-function ultrasound devices, the ultrasonic couplant can be a solid-like couplant

In an embodiment of any of the foregoing dual-function ultrasound devices, the ultrasonic couplant can be a hydrogel

In an embodiment of any of the foregoing dual-function ultrasound devices, a portion of the ultrasonic couplant can be exposed at a bottom opening in the frame.

In an embodiment of any of the foregoing dual-function ultrasound devices, the portion of the ultrasonic couplant can protrude outward from the bottom opening in the frame.

In an embodiment of any of the foregoing dual-function ultrasound devices, the portion of the ultrasonic couplant can have a coefficient of friction on skin of less than 0.1.

In an embodiment of any of the foregoing dual-function ultrasound devices, the ultrasound transducer can include a sensor head configured to be received in a top opening in the frame.

In an embodiment of any of the foregoing dual-function ultrasound devices, the adherable couplant can be disposed on the sensor head.

In an embodiment of any of the foregoing dual-function ultrasound devices, the adherable couplant can be disposed between the sensor head and the ultrasonic couplant.

In an embodiment of any of the foregoing dual-function ultrasound devices, wherein the frame can be detachably coupled to the ultrasound transducer by magnets, or a clip, or a combination thereof.

In an embodiment of any of the foregoing dual-function ultrasound devices, the frame can be configured to slide away from the ultrasound transducer upon detachment.

In an embodiment of any of the foregoing dual-function ultrasound devices, the frame can be configured to be lowered from the ultrasound transducer upon detachment.

In an embodiment of any of the foregoing dual-function ultrasound devices, the frame can further include a film liner. The film liner can be disposed between the ultrasonic couplant and the adherable couplant. The film liner can be configured to detach from the adherable couplant upon detachment of the frame from the ultrasound transducer.

In an embodiment of any of the foregoing dual-function ultrasound devices, the ultrasound transducer can include a protective sheath.

In an embodiment of any of the foregoing dual-function ultrasound devices, the protective sheath can be attached to a sensor head of the ultrasound transducer.

In an embodiment of any of the foregoing dual-function ultrasound devices, the protective sheath can be attached adjacent to the adherable couplant on a face of the sensor head.

In an embodiment of any of the foregoing dual-function ultrasound devices, the protective sheath can form a portion of the adherable couplant.

In an embodiment of any of the foregoing dual-function ultrasound devices, the ultrasound transducer can include a sensor head having a sensor face, the sensor face coupled to the ultrasonic couplant; and a cable extending from the sensor head parallel to the sensor face.

In an embodiment of any of the foregoing dual-function ultrasound devices, the ultrasound transducer can have a thin profile with a height to width ratio of less than half.

A device for use with an ultrasound transducer includes a frame, an ultrasonic couplant disposed in the frame, an adherable couplant disposed on the ultrasonic couplant. The ultrasonic couplant and the adherable couplant are capable of transmitting ultrasound energy. The adherable couplant is configured to interface with the ultrasound transducer. The ultrasonic couplant is configured to interface with the skin of a patient.

The device of the preceding paragraph can optionally include, additionally and/or alternatively, any one or more of the following features, configurations and/or additional components:

In an embodiment of the foregoing device, the adherable couplant can include an adhesive hydrogel.

In an embodiment of any of the foregoing devices, the adherable couplant can include a reinforcing scrim.

In an embodiment of any of the foregoing devices, the adherable couplant can include a first adhesive layer configured to adhere the adherable couplant to a face of the ultrasound transducer; a second adhesive layer configured to adhere the ultrasound transducer to the skin; and a film liner disposed between the first and second adhesive layers. Each of the first and second adhesive layers and the film liner can be capable of transmitting ultrasonic energy.

In an embodiment of any of the foregoing devices, the adherable couplant can include a thermoplastic elastomer.

In an embodiment of any of the foregoing devices, the ultrasonic couplant can include a hydrogel.

In an embodiment of any of the foregoing devices, the frame can include an attachment mechanism configured to interact with and retain the ultrasound transducer.

In an embodiment of any of the foregoing devices, the attachment mechanism can include a magnet, or a clip, or a combination thereof.

In an embodiment of any of the foregoing devices, the frame can include an ejection element configured to release the ultrasound transducer from the frame upon detachment.

A further embodiment of any of the foregoing devices can include a film liner disposed between the adherable couplant and the ultrasonic couplant. The film liner can be capable of transmitting ultrasound energy.

In an embodiment of any of the foregoing devices, a portion of the ultrasonic couplant can protrude through a bottom opening in the frame.

An embodiment of any of the foregoing devices can further include a first seal disposed on surface of the portion of the ultrasonic couplant that protrudes through the bottom opening.

An embodiment of any of the foregoing devices can further include a second seal disposed on the adherable couplant.

In an embodiment of any of the foregoing devices, the first seal can be affixed to a surface of packaging container.

In an embodiment of any of the foregoing devices, the second seal can be affixed to a lid of the packing container.

A method of using a dual-function ultrasound device includes attaching an ultrasound transducer to a frame including an ultrasonic couplant, scanning, via the ultrasound transducer and the ultrasonic couplant, an area on a human body to identify a target location for ultrasound monitoring, detaching the frame including the ultrasonic couplant from the ultrasound transducer, and adhering the ultrasound transducer to the target location for ultrasound monitoring of the target location.

The method of the preceding paragraph can optionally include, additionally and/or alternatively, any one or more of the following features, configurations, additional components, and/or steps:

In an embodiment of the foregoing method, detaching the frame from the ultrasound transducer can include maintaining a location of the ultrasound transducer above the target location.

In an embodiment of any of the foregoing methods, detaching the frame from the ultrasound transducer can include sliding the frame away from the ultrasound transducer or lifting the ultrasound transducer from the frame.

In an embodiment of any of the foregoing methods, adhering the ultrasound transducer to the target location can include pressing the ultrasound transducer onto skin in the target location, wherein the ultrasound transducer comprises an adherable couplant.

In an embodiment of any of the foregoing methods, detaching the frame from the ultrasound transducer can include separating the ultrasonic couplant from the adherable couplant.

In an embodiment of any of the foregoing methods, the adherable couplant can include an adhesive hydrogel capable of transmitting ultrasonic energy.

In an embodiment of any of the foregoing methods, the adherable couplant can include a reinforcing scrim capable of transmitting ultrasonic energy.

In an embodiment of any of the foregoing methods, the ultrasonic couplant can include a solid-like couplant material.

In an embodiment of any of the foregoing methods, the ultrasonic couplant can be a hydrogel.

In an embodiment of any of the foregoing methods, adhering the ultrasound transducer to the target location can further include applying an adhesive patch over the ultrasound transducer and adjacent skin.

The above method(s) can be performed on a living animal or on a simulation, such as on a cadaver, cadaver heart, anthropomorphic ghost, simulator (e.g., with body parts, heart, tissue, etc. being simulated).

While the invention has been described with reference to an exemplary embodiment(s), it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A dual-function ultrasound device for ultrasonic scanning and use as a wearable ultrasonic patch, the dual-function ultrasound device comprising:
an ultrasound transducer;
a detachable frame coupled to the ultrasound transducer, the detachable frame comprising an ultrasonic couplant; and
a protective barrier configured to isolate the ultrasound transducer from biological contamination.

2. The dual-function ultrasound device of claim 1, wherein the ultrasound transducer comprises a sensor head configured to be received in a first opening in the detachable frame, and wherein the sensor head comprises a first attachment mechanism and the detachable frame comprises a second attachment mechanism, the first and second attachment mechanisms configured to interact to retain the sensor head in the detachable frame.

3. The dual-function ultrasound device of claim 2, wherein the detachable frame comprises a second opening opposite the first opening, wherein the ultrasonic couplant protrudes outward from the second opening.

4. The dual-function ultrasound device of claim 3, wherein walls of the detachable frame taper inward toward the second opening.

5. The dual-function ultrasound device of claim 1, wherein the protective barrier comprises:
a film disposed in the detachable frame, the film separating a layer of the ultrasonic couplant from the ultrasound transducer; and
a sheath coupled to the film and extending around a sensor head and at least a portion of a cable of the ultrasound transducer.

6. A device for use with an ultrasound transducer, the device comprising:
a frame;
an ultrasonic couplant disposed in the frame; and
an adherable couplant disposed on the ultrasonic couplant;
wherein the ultrasonic couplant and the adherable couplant are capable of transmitting ultrasound energy; and
wherein the adherable couplant is configured to interface with the ultrasound transducer and wherein the ultrasonic couplant is configured to interface with skin of a patient.

7. The device of claim 6, wherein the adherable couplant comprises an adhesive hydrogel.

8. The device of claim 7, wherein the adherable couplant comprises a reinforcing scrim.

9. The device of claim 7, wherein the adherable couplant comprises:
a first adhesive layer configured to adhere the adherable couplant to a face of the ultrasound transducer;
a second adhesive layer configured to adhere the ultrasound transducer to the skin; and
a film liner disposed between the first and second adhesive layers;
wherein each of the first and second adhesive layers and the film liner is capable of transmitting ultrasonic energy.

10. The device of claim 6, wherein the frame comprises:
an attachment mechanism configured to interact with and retain the ultrasound transducer, wherein the attachment mechanism includes a magnet, or a clip, or a combination thereof; and
an ejection element configured to release the ultrasound transducer from the frame upon detachment.

11. The device of claim 6, and further comprising a film liner disposed between the adherable couplant and the ultrasonic couplant and configured to detach from the adherable couplant upon detachment of the frame from the ultrasound transducer, wherein the film liner is capable of transmitting ultrasound energy.

12. The device of claim 6, wherein the ultrasonic couplant is a hydrogel and wherein a portion of the ultrasonic couplant protrudes through a bottom opening in the frame.

13. The device of claim 12, and further comprising a first seal disposed on surface of the portion of the ultrasonic couplant that protrudes through the bottom opening, wherein the first seal is affixed to a surface of a packaging container.

14. The device of claim 13, and further comprising a second seal disposed on the adherable couplant, wherein the second seal is affixed to a lid of the packing container.

15. The device of claim 6, wherein the adherable couplant further comprises a protective sheath configured to extend around a sensor head and cable of the ultrasound transducer to isolate the ultrasound transducer from biological contamination.

16. A method of using a dual-function ultrasound device, the method comprising:
attaching an ultrasound transducer to a frame, wherein the frame contains an ultrasonic couplant;
scanning, via the ultrasound transducer and the ultrasonic couplant, an area on a human body to identify a target location for ultrasound monitoring;
detaching the frame including the ultrasonic couplant from the ultrasound transducer; and
adhering the ultrasound transducer to the target location for ultrasound monitoring of the target location.

17. The method of claim 16, wherein detaching the frame from the ultrasound transducer comprises sliding the frame away from the ultrasound transducer or lifting the ultrasound transducer from the frame.

18. The method of claim 16, wherein adhering the ultrasound transducer to the target location comprises pressing the ultrasound transducer onto skin in the target location, wherein the ultrasound transducer comprises an adherable couplant capable of transmitting ultrasonic energy.

19. The method of claim 18, wherein detaching the frame from the ultrasound transducer comprises separating the ultrasonic couplant from the adherable couplant.

20. The method of claim 16, wherein adhering the ultrasound transducer to the target location further comprises applying an adhesive patch over the ultrasound transducer and adjacent skin.
